(19) United States
(12) Patent Application Publication
(10) Pub. No.: US 2011/0059154 A1
(43) Pub. Date: Mar. 10, 2011

(54) CCR5 ANTAGONISTS AS PROPHYLACTICS FOR PREVENTING HIV INFECTION AND METHODS OF INHIBITING TRANSMISSION OF SAME

(76) Inventor: Julie M. Strizki, Yardley, PA (US)

(21) Appl. No.: 12/919,833

(22) PCT Filed: Feb. 24, 2009
(86) PCT No.: PCT/US09/34990

§ 371 Date: Nov. 29, 2010

Related U.S. Application Data
(60) Provisional application No. 61/032,539, filed on Feb. 29, 2008.

(51) Int. Cl. A61F 6/06; C07D 401/14; A61K 31/4545; C07D 403/04; A61K 31/506; A61P 31/18

(52) U.S. Cl. 424/430; 546/187; 514/316; 546/189; 544/335; 514/256; 544/295; 514/252.18

(57) ABSTRACT

Topical cream, ointment, lotion, gel, foam formulations and slow release formulations or devices are provided including certain CCR5 antagonists as prophylactics for the prevention, retardation or inhibition of transmission of Human Immunodeficiency Virus (HIV) infection from one human to another.

### FIELD OF THE INVENTION

The present invention relates to topical formulations or preparations comprising certain CCR5 antagonists as prophylactics for Human Immunodeficiency Virus (“HIV”) infection, such as vaginal or rectal topical preparations for use in preventing HIV infection or inhibiting transmission of HIV.

### BACKGROUND OF THE INVENTION

Citation of any reference in this section or any other section in this application shall not be deemed an admission that such reference is available as prior art to this application.

The lack of proper medicaments to prevent and/or stop the spread of HIV among sexual partners has been well documented. See, for example, (1) J. Cohen, Science, (Oct. 15, 2004) 306, page 387 (“Microbicides have long had a stepchild status in the AIDS research community. Industry has had little interest in developing a topical gel or cream that can stop HIV at the vagina or rectum, and the products that have moved furthest in human studies are soaps and other substances that do not specifically target the virus.”); (2) J. Turpin, Expert. Opin. Investig. Drugs, (August 2002) 11(8), 1077-1097 (“The increased incidence of HIV/AIDS disease in women aged 15-49 years has identified the urgent need for a female-controlled, efficacious and safe vaginal topical microbicide.”); (3) R. Trager, Science, (Jan. 3, 2003), 299 39 (“Researchers hope to create pathogen-stopping gels or creams that can be used before sexual intercourse. They would not be a substitute for condoms, but they might give extra protection at low cost—particularly important in the developing world. And they might offer women a new method of blocking disease that doesn't depend on male cooperation. Although the idea has been around for many years, no microbicide has yet proved clinically effective against HIV.”).

Infection with HIV leads to Acquired Immunodeficiency Syndrome (“AIDS”) or AIDS Related Complex (“ARC”) in over 90% of untreated infected individuals within a ten-year period. As the HIV epidemic continues to threaten millions of people worldwide, new strategies to prevent the spread of the virus are desperately needed. In the absence of an effective preventative vaccine, alternative methods of preventing HIV infection are currently being explored. Among these is the use of topical microbicide preparations that can inactivate infectious agents in contaminated fluids or block infection of susceptible cells at the site of transmission. To date, a variety of peptide and polymer based microbicides have proven active in in vitro infection systems and some of these have shown activity in in vivo infection models.

Thus, for example, the above-cited Science (Jul. 8, 2004) article refers to certain detergent-based, carbohydrate-based or polymer-based microbicides that have been tested or are in the process of being tested, as well as their advantages and disadvantages.

C. C. Tsai et al, AIDS Res. Hum. Retroviruses (July 2003) 19(7) 535-541 describe the testing of cyanovirin-N gel as a topical microbicide to prevent rectal transmission of SHIV viruses.

A. Neurath et al, BMC Infect. Dis. (Oct. 14, 2004) 4(1), 41 describe the use of Punica granatum (pomegranate) juice as an HIV-entry preventer.

HIV typically establishes an infection by first attaching to CD4 receptors on white blood cells and then grabbing a second receptor known as CC Chemokine Receptor 5 (“CCR5”), which normally responds to immune chemicals called chemokines. Epidemiological and viral transmission studies have shown that viruses using the CCR5 receptor are often associated with transmission of HIV infection between individuals. Therefore blocking these viruses by prophylactic treatment with a specific CCR5 inhibitor should prove an effective way to prevent HIV transmission in a susceptible population. For example, M. Lederman et al, Science (Oct. 15, 2004) 306, 485-487 describe a study of the ability of N^{α}-(n-nonanoyl)-des-Ser¹-[L-thioproline², L-α-cyclohexyl-glycine³] RANTES (“PSC-RANTES”) to prevent acquisition of SHIV infection at a mucosal membrane. Q. Hu et al, J. Exp. Med. (Apr. 19, 2004) 199(8), 1065-1075 describe the blockade of the effect of both CCR5 and CXCR4 to prevent infection.

However the development of some of these early microbicides may be limited by factors such as limited absorption, irritation or disruption of mucosal tissues, agent stability or cost. Therefore, small molecule inhibitors with better pharmacokinetic stability and toxicity profiles may provide a more attractive alternative to the peptide or polymer-based microbicides. See, Veazey et al, Nature (Nov. 3, 2005) 438, 99-102) “Protection of Macaques from vaginal SHIV challenge by vaginally delivered inhibitors of virus-cell fusion”.

Thus, there is an urgent need to provide easily applicable, preferably but not limited to, female-controlled, formulations and methods of local or systemic treatment based on small molecule CCR5 receptor antagonists to prevent or inhibit the transmission of HIV. There is also a need to provide such methods of prevention or inhibition that can be used without partner consent. As can be understood, a suitable method can be useful for preventing viral infection, either vaginally or rectally or both.

### SUMMARY OF THE INVENTION

This invention provides topical formulations or preparations, for example, a cream, gel, ointment, lotion, foam, tablet or film, or a vaginal device (such as a vaginal ring device, an IUD or a sponge), and the like, comprising at least one small molecule CCR5 receptor antagonist.

In some embodiments, the topical formulations are suitable for vaginal, rectal or buccal administration.

In some embodiments of the present invention, the CCR5 receptor antagonist is:

(i) a compound represented by the structural Formula I:

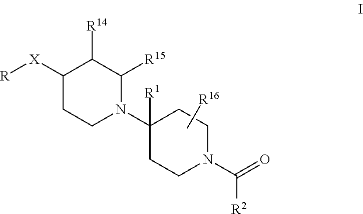

or a pharmaceutically acceptable salt thereof, wherein

X is —C(R¹³)₂—, —C(R¹³)(R¹⁹)—, —C(O)—, —O—, —NH—, —N((C₁-C₆)alkyl)-,

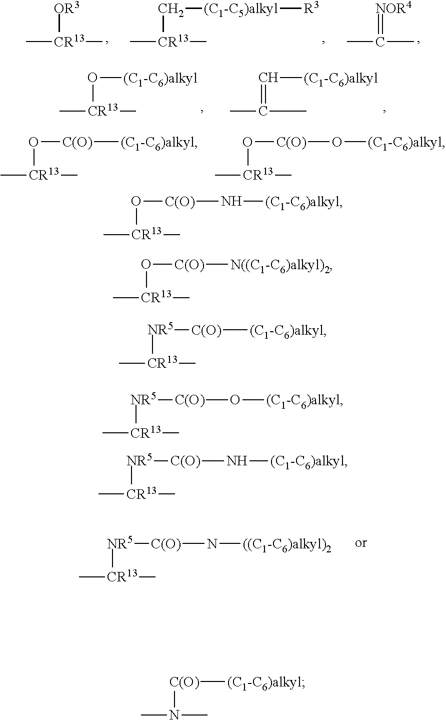

R is R⁶-phenyl, R⁶-pyridyl, R⁶-thiophenyl or R⁶-naphthyl;

R¹ is hydrogen, C₁-C₆ alkyl or C₂-C₆ alkenyl;

R² is R⁷, R⁸, R⁹-phenyl; R⁷, R⁸, R⁹-substituted 6-membered heteroaryl; R⁷, R⁸, R⁹-substituted 6-membered heteroaryl N-oxide; R¹⁰, R¹¹-substituted 5-membered heteroaryl; naphthyl; fluorenyl; diphenylmethyl

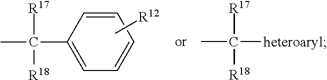

R³ is R⁶-phenyl, R⁶-heteroaryl or R⁶-naphthyl;

R⁴ is hydrogen, C₁-C₆ alkyl, fluoro-C₁-C₆ alkyl, cyclopropylmethyl,

—CH₂CH₂OH, —CH₂CH₂—O—(C₁-C₆)alkyl, —CH₂C(O)—O—(C₁-C₆)alkyl, —CH₂C(O)NH₂, —CH₂C(O)—NH(C₁-C₆)alkyl or —CH₂C(O)—N((C₁-C₆)alkyl)₂;

R⁵ and R¹¹ are independently selected from the group consisting of hydrogen and (C₁-C₆)-alkyl;

R⁶ is 1 to 3 substituents independently selected from the group consisting of hydrogen, halogen, C₁-C₆ alkyl, C₁-C₆ alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃)—, CH₃C(═NOCH₂CH₃)—,

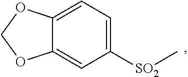

—NH₂, —NHCOCF₃, —NHCONH(C₁-C₆ alkyl), —NHCO(C₁-C₆ alkyl), —NHSO₂(C₁-C₆ alkyl), 5-membered heteroaryl and

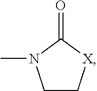

wherein X is —O—, —NH— or —N(CH₃)—;

R⁷ and R⁸ are independently selected from the group consisting of (C₁-C₆)alkyl, halogen, —NR²⁰R²¹, —OH, —CF₃, —OCH₃, —O-acyl, and —OCF₃;

R⁹ is R⁷, hydrogen, phenyl, —NO₂, —CN, —CH₂F, —CHF₂, —CHO, —CH═NOR²⁰, pyridyl, pyridyl N-oxide, pyrimidinyl, pyrazinyl, —N(R²⁰)CONR²¹R²², —NHCONH(chloro-(C₁-C₆)alkyl), —NHCONH((C₃-C₁₀-cycloalkyl(C₁-C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂CF₃)₂, —NHCO₂(C₁-C₆)alkyl, C₃-C₁₀ cycloalkyl, —SR²³, —SOR²³, —SO₂R²³, —SO₂NH(C₁-C₆ alkyl), —OSO₂(C₁-C₆)alkyl, —OSO₂CF₃, hydroxy(C₁-C₆)alkyl, —CONR²⁰R²¹, —CON(CH₂CH₂—O—CH₃)₂, —OCONH(C₁-C₆)alkyl, —CO₂R²⁰, —Si(CH₃)₃ or —B(OC(CH₃)₂)₂;

R¹⁰ is (C₁-C₆)alkyl, —NH₂ or R¹²-phenyl;

R¹² is 1 to 3 substituents independently selected from the group consisting of hydrogen, (C₁-C₆)alkyl, —CF₃, —CO₂R₂₀, —CN, (C₁-C₆)alkoxy and halogen;

R¹³, R¹⁴, R¹⁵ and R¹⁶ are independently selected from the group consisting of hydrogen and (C₁-C₆)alkyl;

R¹⁷ and R¹⁸ are independently selected from the group consisting of hydrogen and C₁-C₆ alkyl, or R¹⁷ and R¹⁸ together are a C₂-C₅ alkylene group and with the carbon to which they are attached form a spiro ring of 3 to 6 carbon atoms;

R¹⁹ is R⁶-phenyl, R⁶-heteroaryl, R⁶-naphthyl, C₃-C₁₀ cycloalkyl, (C₃-C₁₀)cycloalkyl(C₁-C₆)alkyl or (C₁-C₆)alkoxy(C₁-C₆)alkyl;

R²⁰, R²¹ and R²² are independently selected from the group consisting of H and C₁-C₆ alkyl; and

R²³ is C₁-C₆ alkyl or phenyl;

(ii) a compound represented by the structural Formula II

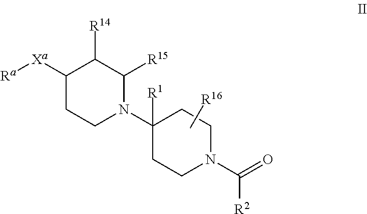

or a pharmaceutically acceptable salt thereof, wherein
(1) X^{a} is —C(R¹³)₂—, —C(R¹³)(R¹⁹)—, —NH—, —N((C₁-C₆)alkyl)-,

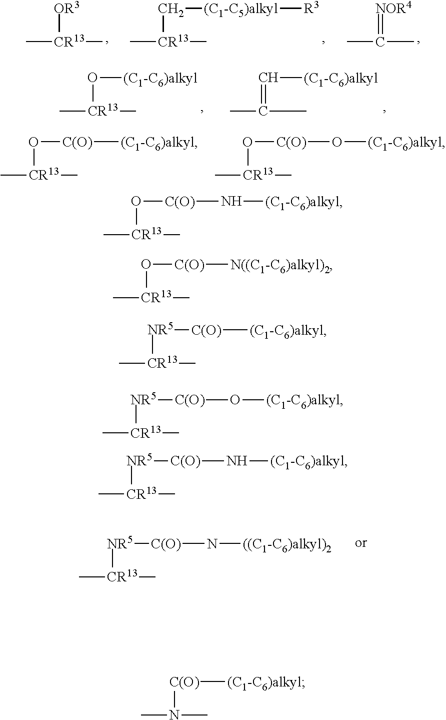

R^{a} is R^{6a}-phenyl, R^{6a}-pyridyl, R^{6a}-thiophenyl or R⁶-naphthyl;

R¹ is hydrogen, C₁-C₆ alkyl or C₂-C₆ alkenyl;

R² is R⁷, R⁸, R⁹-phenyl; R⁷, R⁸, R⁹-substituted 6-membered heteroaryl; R⁷, R⁸, R⁹-substituted 6-membered heteroaryl N-oxide; R¹⁰, R¹¹-substituted 5-membered heteroaryl; naphthyl; fluorenyl; diphenylmethyl

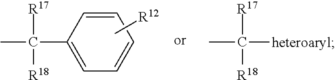

R³ is R¹⁰-phenyl, pyridyl, pyrimidyl, pyrazinyl or thiazolyl;

R⁴ is hydrogen, C₁-C₆ alkyl, fluoro-C₁-C₆ alkyl, cyclopropylmethyl,

—CH₂CH₂OH, —CH₂CH₂—O—(C₁-C₆)alkyl, —CH₂C(O)—O—(C₁-C₆)alkyl, —CH₂C(O)NH₂, —CH₂C(O)—NH(C₁-C₆)alkyl or —CH₂C(O)—N((C₁-C₆)alkyl)₂;

R⁵ and R¹¹ are independently selected from the group consisting of hydrogen and (C₁-C₆)-alkyl;

R^{6a} is 1 to 3 substituents independently selected from the group consisting of hydrogen, halogen, —CF₃, CF₃O—, —CN, —CF₃SO₂—, R¹²-phenyl, —NHCOCF₃, 5-membered heteroaryl and

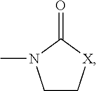

wherein X is —O—, —NH— or —N(CH₃)—;

R⁶ is independently selected from the group consisting of R^{6a} and CH₃SO₂—;

R⁷ and R⁸ are independently selected from the group consisting of (C₁-C₆)alkyl, halogen, —NR²⁰R²¹, —OH, —CF₃, —OCH₃, —O-acyl, and —OCF₃;

R⁹ is R⁷, hydrogen, phenyl, —NO₂, —CN, —CH₂F, —CHF₂, —CHO, —CH═NOR²⁰, pyridyl, pyridyl N-oxide, pyrimidinyl, pyrazinyl, —N(R²⁰)CONR²¹R²², —NHCONH(chloro-(C₁-C₆)alkyl), —NHCONH((C₃-C₁₀)-cycloalkyl(C₁-C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂CF₃)₂, —NHCO₂(C₁-C₆)alkyl, C₃-C₁₀ cycloalkyl, —SR²³, —SOR²³, —SO₂R²³, —SO₂NH(C₁-C₆ alkyl), —OSO₂(C₁-C₆)alkyl, —OSO₂CF₃, hydroxy(C₁-C₆)alkyl, —CONR²⁰R²¹, —CON(CH₂CH₂—O—CH₃)₂, —OCONH(C₁-C₆)alkyl, —CO₂R²⁰, —Si(CH₃)₃ or —B(OC(CH₃)₂)₂;

R¹⁰ is (C₁-C₆)alkyl, —NH₂ or R¹²-phenyl;

R¹² is 1 to 3 substituents independently selected from the group consisting of hydrogen, (C₁-C₆)alkyl, —CF₃, —CO₂R₂₀, —CN, (C₁-C₆)alkoxy and halogen;

R¹³, R¹⁴, R¹⁵ and R¹⁶ are independently selected from the group consisting of hydrogen and (C₁-C₆)alkyl;

R¹⁷ and R¹⁸ are independently selected from the group consisting of hydrogen and C₁-C₆ alkyl, or R¹⁷ and R¹⁶ together are a C₂-C₅ alkylene group and with the carbon to which they are attached form a spiro ring of 3 to 6 carbon atoms;

R¹⁹ is R⁶-phenyl, R⁶-heteroaryl, R⁶-naphthyl, C₃-C₁₀ cycloalkyl, (C₃-C₁₀)cycloalkyl(C₁-C₆)alkyl or (C₁-C₆)alkoxy(C₁-C₆)alkyl;

R²⁰, R²¹ and R²² are independently selected from the group consisting of H and C₁-C₆ alkyl; and

R²³ is C₁-C₆ alkyl or phenyl;

or

(2): X^{a} is —C(R¹³)(R¹⁹)—, —C(O)—, —O—, —NH—, —N((C₁-C₆)alkyl)-,

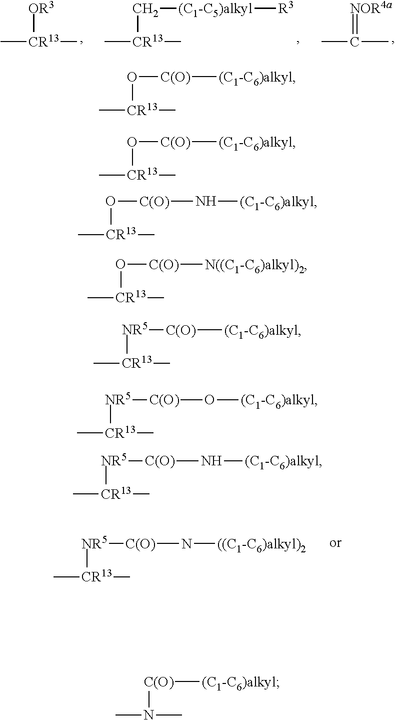

R^{a} is R^{6b}-phenyl, R^{6b}-pyridyl or R^{6b}-thiophenyl;

R^{4a} is fluoro-C₁-C₆ alkyl, cyclopropylmethyl, —CH₂CH₂OH, —CH₂CH₂—O—(C₁-C₆)alkyl, —CH₂C(O)—O—(C₁-C₆)alkyl, —CH₂C(O)NH₂, —CH₂C(O)—NH—(C₁-C₆)alkyl or —CH₂C(O)—N((C₁-C₆)alkyl)₂;

R^{6b} is CH₃SO₂—; and

R¹, R², R³, R⁶, R¹⁴, R¹⁵, R¹⁶ and R¹⁹ are as defined in (1) above;

(iii) a compound represented by the structural Formula III:

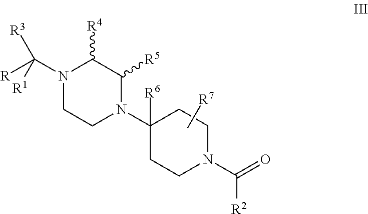

or a pharmaceutically acceptable salt thereof, wherein

R is R⁸-phenyl, R⁸-pyridyl, R⁸-thiophenyl or R⁸-naphthyl;

R¹ is hydrogen or C₁-C₆ alkyl;

R² is R⁹, R¹⁰, R¹¹-phenyl; R⁹, R¹⁰, R¹¹-substituted 6-membered heteroaryl; R⁹, R¹⁰, R¹¹-substituted 6-membered heteroaryl N-oxide; R¹², R¹³-substituted 5-membered heteroaryl; naphthyl; fluorenyl; diphenylmethyl

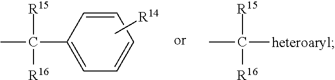

R³ is hydrogen, C₁-C₆ alkyl, (C₁-C₆)alkoxy(C₁-C₆)alkyl, C₃-C₁₀ cycloalkyl, C₃-C₁₀ cycloalkyl(C₁-C₆)alkyl, R⁸-phenyl, R⁸-phenyl(C₁-C₆)alkyl, R⁸-naphthyl, R⁸-naphthyl(C₁-C₆)alkyl, R⁸-heteroaryl or R⁸-heteroaryl(C₁-C₆)alkyl;

R⁴, R⁵, R⁷ and R¹³ are independently selected from the group consisting of hydrogen and (C₁-C₆)-alkyl;

R6 is hydrogen, C₁-C₆ alkyl or C₂-C₆ alkenyl;

R⁸ is 1 to 3 substituents independently selected from the group consisting of hydrogen, halogen, C₁-C₆ alkyl, C₁-C₆ alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃), CH₃C(═NOCH₂CH₃),

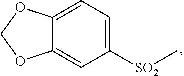

—NH₂, —NHCOCF₃, —NHCONH(C₁-C₆ alkyl), —NHCO(C₁-C₆ alkyl), —NHSO₂(C₁-C₆ alkyl), 5-membered heteroaryl and

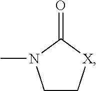

wherein X is —O—, —NH— or —N(CH₃)—;

R⁹ and R¹⁰ are independently selected from the group consisting of (C₁-C₆)alkyl, halogen, —NR¹⁷R¹⁸, —OH, —CF₃, —OCH₃, —O-acyl, —OCF₃ and —Si(CH₃)₃;

R¹¹ is R⁹, hydrogen, phenyl, —NO₂, —CN, —CH₂F, —CHF₂, —CHO, —CH═NOR¹⁷, pyridyl, pyridyl N-oxide, pyrimidinyl, pyrazinyl, —N(R¹⁷)CONR¹⁸R¹⁹, —NHCONH(chloro-(C₁-C₆)alkyl), —NHCONH((C₃-C₁)cycloalkyl(C₁-C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂CF₃)₂, —NHCO₂(C₁-C₆)alkyl, C₃-C₁₀ cycloalkyl, —SR²⁰, —SOR²⁰, —SO₂R²⁰, —SO₂NH(C₁-C₆ alkyl), —OSO₂(C₁-C₆)alkyl, —OSO₂CF₃, hydroxy(C₁-C₆)alkyl, —CONR¹⁷R¹⁸, —CON(CH₂CH₂—O—CH₃)₂, —OCONH(C₁-C₆)alkyl, —CO₂R¹⁷, —Si(CH₃)₃ or —B(OC(CH₃)₂)₂;

R¹² is (C₁-C₆)alkyl, —NH₂ or R¹⁴-phenyl;

R¹⁴ is 1 to 3 substituents independently selected from the group consisting of hydrogen, (C₁-C₆)alkyl, —CF₃, —CO₂R₁₇, —CN, (C₁-C₆)alkoxy and halogen;

R¹⁵ and R¹⁶ are independently selected from the group consisting of hydrogen and C₁-C₆ alkyl, or R¹⁵ and R¹⁶ together are a C₂-C₅ alkylene group and with the carbon to which they are attached form a spiro ring of 3 to 6 carbon atoms;

R¹⁷, R¹⁸ and R¹⁹ are independently selected from the group consisting of H and C₁-C₆ alkyl; and

R²⁰ is C₁-C₆ alkyl or phenyl;

(iv) a compound represented by the structural Formula IV

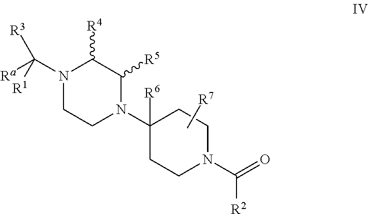

or a pharmaceutically acceptable salt thereof, wherein
(1) R^{a} is R^{8a}-phenyl, R^{8b}-pyridyl, R^{8b}-thiophenyl or R⁸-naphthyl;

R¹ is hydrogen or C₁-C₆ alkyl;

R² is R⁹, R¹⁰, R¹¹-phenyl; R⁹, R¹⁰, R¹¹-substituted 6-membered heteroaryl; R⁹, R¹⁰, R¹¹-substituted 6-membered heteroaryl N-oxide; R¹², R¹³-substituted 5-membered heteroaryl; naphthyl; fluorenyl; diphenylmethyl,

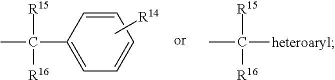

R³ is hydrogen, C₁-C₆ alkyl, (C₁-C₆)alkoxy(C₁-C₆)alkyl, C₃-C₁₀ cycloalkyl, C₃-C₁₀ cycloalkyl(C₁-C₆)alkyl, R⁸-phenyl, R⁸-phenyl(C₁-C₆)alkyl, R⁸-naphthyl, R⁸-naphthyl(C₁-C₆)alkyl, R⁸-heteroaryl or R⁸-heteroaryl(C₁-C₆)alkyl;

R⁴, R⁵, R⁷ and R¹³ are independently selected from the group consisting of hydrogen and (C₁-C₆)-alkyl;

R⁶ is hydrogen, C₁-C₆ alkyl or C₂-C₆ alkenyl;

R⁸ is 1 to 3 substituents independently selected from the group consisting of hydrogen, halogen, C₁-C₆ alkyl, C₁-C₆ alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃), CH₃C(═NOCH₂CH₃),

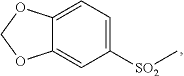

—NH₂, —NHCOCF₃, —NHCONH(C₁-C₆ alkyl), —NHCO(C₁-C₆ alkyl), —NHSO₂(C₁-C₆ alkyl), 5-membered heteroaryl and

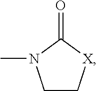

wherein X is —O—, —NH— or —N(CH₃)—;

R^{8a} is 1 to 3 substituents independently selected from the group consisting of hydrogen, halogen, —CF₃, CF₃O—, —CN, CF₃SO₂—, R¹⁴-phenyl, —NHCOCF₃, 5-membered heteroaryl and

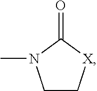

wherein X is as defined above;

R^{8b} is 1 to 3 substituents independently selected from the group consisting of hydrogen, halogen, —CF₃, CF₃O—, CH₃C(O)—, —CN, CF₃SO₂—, R¹⁴-benzyl, CH₃C(═NOCH₃), CH₃C(═NOCH₂CH₃),

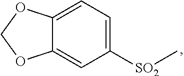

—NHCOCF₃, 5-membered heteroaryl and

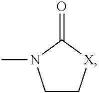

wherein X is as defined above;

R⁹ and R¹⁰ are independently selected from the group consisting of (C₁-C₆)alkyl, halogen, —NR¹⁷R¹⁸, —OH, —CF₃, —OCH₃, —O-acyl, —OCF₃ and —Si(CH₃)₃;

R¹¹ is R⁹, hydrogen, phenyl, —NO₂, —CN, —CH₂F, —CHF₂, —CHO, —CH═NOR¹⁷, pyridyl, pyridyl N-oxide, pyrimidinyl, pyrazinyl, —N(R¹⁷)CONR¹⁸R¹⁹, —NHCONH(chloro-(C₁-C₆)alkyl), —NHCONH((C₃-C₁)cycloalkyl(C₁-C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂CF₃)₂, —NHCO₂(C₁-C₆)alkyl, C₃-C₁₀ cycloalkyl, —SR²⁰, —SOR²⁰, —SO₂R²⁰, —SO₂NH(C₁-C₆ alkyl), —OSO₂(C₁-C₆)alkyl, —OSO₂CF₃, hydroxy(C₁-C₆)alkyl, —CONR¹⁷R¹⁸, —CON(CH₂CH₂—O—CH₃)₂, —OCONH(C₁-C₆)alkyl, —CO₂R¹⁷, —Si(CH₃)₃ or—B(OC(CH₃)₂)₂;

R¹² is (C₁-C₆)alkyl, —NH₂ or R¹⁴-phenyl;

R¹⁴ is 1 to 3 substituents independently selected from the group consisting of hydrogen, (C₁-C₆)alkyl, —CF₃, —CO₂R₁₇, —CN, (C₁-C₆)alkoxy and halogen;

R¹⁵ and R¹⁶ are independently selected from the group consisting of hydrogen and C₁-C₆ alkyl, or R¹⁵ and R¹⁶ together are a C₂-C₅ alkylene group and with the carbon to which they are attached form a spiro ring of 3 to 6 carbon atoms;

R¹⁷, R¹⁸ and R¹⁹ are independently selected from the group consisting of H and C₁-C₆ alkyl; and

R²⁰ is C₁-C₆ alkyl or phenyl; or

(2) R^{a} is R⁸-phenyl, R⁸-pyridyl or R⁸-thiophenyl;

R² is fluorenyl, diphenylmethyl,

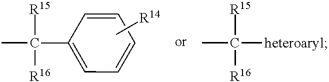

and R¹, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰ are as defined in (1);

(v) a compound represented by the structural Formula V

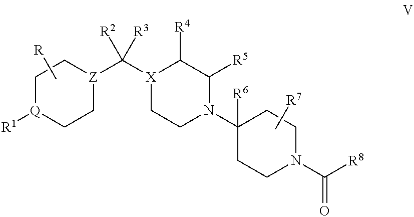

or a pharmaceutically acceptable salt or isomer thereof, wherein:

Q, X and Z are independently selected from the group consisting of CH and N, provided that one or both of Q and Z is N;

R, R⁴, R⁵, R⁶ and R⁷ are independently selected from the group consisting of H and (C₁-C₆)alkyl;

R¹ is H, (C₁-C₆)alkyl, fluoro-(C₁-C₆)alkyl-, R⁹-aryl(C₁-C₆)alkyl-, R⁹-heteroaryl-

(C₁-C₆)alkyl-, (C₁-C₆)alkyl-SO₂—, (C₃-C₆)cycloalkyl-SO₂—, fluoro-(C₁-C₆)alkyl-SO₂—,
R⁹-aryl-SO₂—, R⁹-heteroaryl-SO₂—, N(R²²)(R²³)—SO₂—, (C₁-C₆)alkyl-C(O)—, (C₃-C₆)cyclo-alkyl-C(O)—, fluoro-(C₁-C₆)alkyl-C(O)—, R⁹-aryl-C(O)—, NH—(C₁-C₆)alkyl-C(O)— or R⁹-aryl-NH—C(O)—;

R² is H or (C₁-C₆)alkyl, and R³ is H, (C₁-C₆)alkyl, (C₁-C₆)alkoxy(C₁-C₆)alkyl-,

(C₃-C₁₀)-cycloalkyl-, (C₃-C₁₀)cycloalkyl(C₁-C₆)alkyl-, R⁹-aryl, R⁹-aryl(C₁-C₆)-alkyl-, R⁹-heteroaryl, or R⁹-heteroaryl(C₁-C₆)alkyl-, provided that both X and Z are not each N;

or R² and R³ together are ═O, ═NOR′°, ═N—NR¹¹R¹² or ═CH(C₁-C₆)alkyl, provided that when one or both of X and Z is N, R² and R³ together are not ═CH(C₁-C₆)alkyl;

and when X and Z are each CH, R³ can also be (C₁-C₆)alkoxy, R⁹-aryloxy,

R⁹-heteroaryloxy, (C₁-C₆)alkyl-C(O)O—, (C₁-C₆)alkyl-NH—C(O)O—, N((C₁-C₆)alkyl)₂-C(O)O—, (C₁-C₆)alkyl-C(O)—NR¹³—, (C₁-C₆)alkyl-O—C(O)—NR¹³—, (C₁-C₆)alkyl-NH—C(O)—NR¹³— or N((C₁-C₆)alkyl)₂-C(O)—NR¹³—;

R⁸ is (R¹⁴,R¹⁵,R¹⁶)-substituted phenyl, (R¹⁴,R¹⁵,R¹⁶)-substituted 6-membered heteroaryl, (R¹⁴,R¹⁵,R¹⁶)-substituted 6-membered heteroaryl N-oxide, (R¹⁷,R¹⁸)-substituted 5-membered heteroaryl, naphthyl, fluorenyl, diphenylmethyl,

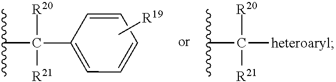

R⁹ is 1, 2 or 3 substituents independently selected from the group consisting of H, halogen, (C₁-C₆)alkyl, (C₁-C₆)alkoxy, —CF₃, —OCF₃, CH₃C(O)—, —CN, CH₃SO₂—, CF₃SO₂— and —N(R²²)(R²³);

R¹⁰ is H, (C₁-C₆)alkyl, fluoro(C₁-C₆)alkyl-, (C₃-C₁₀)cycloalkyl(C₁-C₆)alkyl-, hydroxy(C₂-C₆)alkyl-, (C₁-C₆)alkyl-O—(C₂-C₆)alkyl-, (C₁-C₆)alkyl-O—C(O)—(C₁-C₆)alkyl- or

N(R²²)(R²³)—C(O)—(C₁-C₆)alkyl-,

R¹¹ and R¹² are independently selected from the group consisting of H, (C₁-C₆)alkyl and (C₃-C₁₀)cycloalkyl, or R¹¹ and R¹² together are C₂-C₆ alkylene and form a ring with the nitrogen to which they are attached;

R¹⁴ and R¹⁵ are independently selected from the group consisting of (C₁-C₆)alkyl, halogen, —NR²²R²³, —OH, —CF₃, —OCH₃, —O-acyl and —OCF₃;

R¹⁶ is R¹⁴, hydrogen, phenyl, —NO₂, —CN, —CH₂F, —CHF₂, —CHO, —CH═NOR²⁴, pyridyl, pyridyl N-oxide, pyrimidinyl, pyrazinyl, —N(R²⁴)CONR²⁵R²⁶, —NHCONH(chloro-(C₁-C₆)alkyl), —NHCONH((C₃-C₁₀)cycloalkyl(C₁-C₆)alkyl), —NHCO(C₁-C₆)alkyl, —NHCOCF₃, —NHSO₂N(R²²)(R²³), —NHSO₂(C₁-C₆)alkyl, —N(SO₂CF₃)₂, —NHCO₂—(C₁-C₆)alkyl, C₃-C₁₀ cycloalkyl, —SR²⁷, —SOR²⁷, —SO₂R²⁷, —SO₂NH(R²²), OSO₂(C₁-C₆)alkyl, —OSO₂CF₃, hydroxy(C₁-C₆)alkyl-, —CONR²⁴R²⁵, —CON(CH₂CH₂OCH₃)₂, —OCONH(C₁-C₆)alkyl, —CO₂R²⁴, —Si(CH₃)₃ or—B(OC(CH₃)₂)₂;

R¹⁷ is (C₁-C₆)alkyl, —N(R²²)(R²³) or R¹⁹-phenyl;

R¹³, R¹⁸, R²², R²³, R²⁴, R²⁵ and R²⁶ are independently selected from the group consisting of H and (C₁-C₆)alkyl;

R¹⁹ is 1, 2 or 3 substituents independently selected from the group consisting of H, (C₁-C₆)alkyl, —CF₃, —CO₂R²⁵, —CN, (C₁-C₆)alkoxy and halogen;

R²⁰ and R²¹ are independently selected from the group consisting of H and (C₁-C₆)alkyl, or R²⁰ and R²¹ together with the carbon to which they are attached form a spiro ring of 3 to 6 carbon atoms; and

R²⁷ is (C₁-C₆)alkyl or phenyl

(vi) a compound represented by the structural Formula VI:

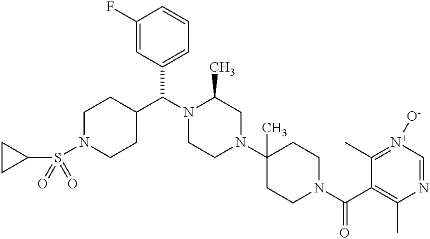

or a pharmaceutically acceptable salt or solvate thereof;

(vii) a compound represented by the structural Formula VII

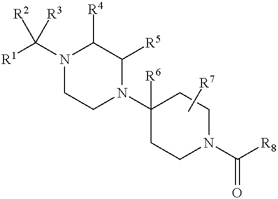

or a pharmaceutically acceptable salt, solvate or ester thereof; wherein:

R¹ is selected from the group consisting of R⁹-phenyl, R⁹-pyridyl, R⁹-thiophenyl, R⁹-naphthyl, and

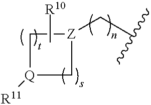

R² is selected from the group consisting of H and alkyl;

R³ is selected from the group consisting of H, alkyl, alkoxyalkyl-, cycloalkyl, cycloalkylalkyl-, R⁹-aryl, R⁹-arylalkyl-, R⁹-heteroaryl, and R⁹-heteroarylalkyl-;

or R² and R³ together are ═O, ═N(OR¹²), or ═N—N(R¹³)(R¹⁴);

R⁴, R⁵, R⁶ and R⁷ are independently selected from the group consisting of H and alkyl;

R⁸ is selected from the group consisting of

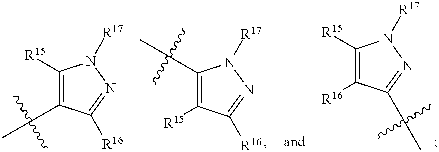

R⁹ is 1, 2 or 3 substituents independently selected from the group consisting of H, halogen, alkyl, alkoxy, —CF₃, —OCF₃, CH₃C(O)—, —CN, CH₃S(O₂)—, CF₃S(O₂)—, —N(R¹⁸)(R¹⁹);

R¹⁰ is selected from the group consisting of H and alkyl;

R¹¹ is selected from the group consisting of H, alkyl, fluoroalkyl-, R⁹-arylalkyl-, R⁹-heteroaryl-, alkyl, alkyl-S(O₂)—, cycloalkyl-S(O₂)—, fluoroalkyl-S(O₂)—, R⁹-aryl-S(O₂)—, R⁹-heteroaryl-S(O₂)—, N(R¹⁸)(R¹⁹)—S(O₂)—, alkyl-C(O)—, cycloalkyl-C(O)—, fluoroalkyl-C(O)—, R⁹-aryl-C(O)—, alkyl-NH—C(O)— and R⁹-aryl-NH—C(O)—;

R¹² is H, alkyl, fluoroalkyl-, cycloalkylalkyl-, hydroxyalkyl-, alkyl-O-alkyl-, alkyl-O—C(O)-alkyl- or N(R¹⁸)(R¹⁹)—C(O)-alkyl-;

R¹³ and R¹⁴ are independently selected from the group consisting of H, alkyl and cycloalkyl, or R¹³ and R¹⁴ together are (C₂-C₆)alkylene and form a ring with the nitrogen atom to which they are shown attached;

R¹⁵ and R¹⁶ are independently selected from the group consisting of alkyl, halogen, —NR¹⁸R¹⁹, —OH, —CF₃, —OCH₃, —O-acyl and —OCF₃;

R¹⁷ is selected from the group consisting of R²⁰O—, H₂N— and R²⁰R²¹N—;

R¹⁸ and R¹⁹ are independently selected from the group consisting of H and alkyl;

R²⁰ is selected from the group consisting of alkyl, haloalkyl cycloalkyl, heterocyclyl, aralkyl, alkylaryl, aryl, and heteroaryl;

R²¹ is selected from the group consisting of H, alkyl, fluoro-alkyl-, R⁹-arylalkyl-, R⁹-heteroaryl-, alkyl, alkyl-S(O₂)—, cycloalkyl-S(O₂)—, fluoroalkyl-S(O₂)—, R⁹-aryl-S(O₂)—, R⁹-heteroaryl-S(O₂)—, N(R¹⁸)(R¹⁹)—S(O₂)—, alkyl-C(O)—, cycloalkyl-C(O)—, fluoroalkyl-C(O)—, R⁹-aryl-C(O)—, alkyl-NH—C(O)— and R⁹-aryl-NH—C(O)—;

Q and Z are independently selected from the group consisting of CH and N;

n is 0, 1, 2, 3 or 4;

s is 0, 1, 2, 3 or 4; and

t is 1, 2, 3 or 4;

with the proviso that when n is 0, Z is CH;

(viii) a compound represented by the structural Formula VIII

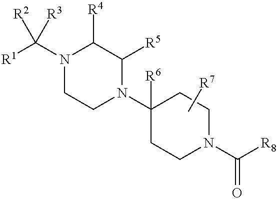

or a pharmaceutically acceptable salt, solvate or ester thereof; wherein:

R¹ is selected from the group consisting of R⁹-phenyl, R⁹-pyridyl, R⁹-thiophenyl, R⁹-naphthyl, and

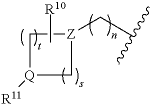

R² is selected from the group consisting of H and alkyl;

R³ is selected from the group consisting of H, alkyl, alkoxyalkyl-, cycloalkyl, cycloalkylalkyl-, R⁹-aryl, R⁹-arylalkyl-, R⁹-heteroaryl, and R⁹-heteroarylalkyl-;

or R² and R³ together are ═O, ═NOR¹², or ═N—N(R¹³)(R¹⁴);

R⁴, R⁵, R⁶ and R⁷ are independently selected from the group consisting of H and alkyl;

R⁸ is selected from the group consisting of

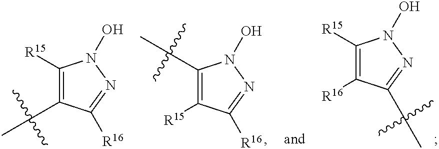

R⁹ is 1, 2 or 3 substituents independently selected from the group consisting of H, halogen, alkyl, alkoxy, —CF₃, —OCF₃, CH₃C(O)—, —CN, CH₃S(O₂)—, CF₃S(O₂)—, —N(R¹⁸)(R¹⁹);

R¹⁰ is selected from the group consisting of H and alkyl;

R¹¹ is selected from the group consisting of H, alkyl, fluoroalkyl-, R⁹-arylalkyl-, R⁹-heteroaryl-, alkyl, alkyl-S(O₂)—, cycloalkyl-S(O₂)—, fluoroalkyl-S(O₂)—, R⁹-aryl-S(O₂)—, R⁹-heteroaryl-S(O₂)—, N(R¹⁸)(R¹⁹)—S(O₂)—, alkyl-C(O)—, cycloalkyl-C(O)—, fluoroalkyl-C(O)—, R⁹-aryl-C(O)—, alkyl-NH—C(O)— and R⁹-aryl-NH—C(O)—;

R¹² is H, alkyl, fluoroalkyl-, cycloalkylalkyl-, hydroxyalkyl-, alkyl-O-alkyl-, alkyl-O—C(O)-alkyl- or N(R¹⁷)(R¹⁸)—C(O)-alkyl-;

R¹³ and R¹⁴ are independently selected from the group consisting of H, alkyl and cycloalkyl, or R¹³ and R¹⁴ together are (C₂-C₆)alkyl and form a ring with the nitrogen atom to which they are shown attached;

R¹⁵ and R¹⁶ are independently selected from the group consisting of alkyl, halogen, —NR¹⁷R¹⁸, —OH, —CF₃, —OCH₃, —O-acyl and —OCF₃;

R¹⁷ and R¹⁸ are independently selected from the group consisting of H and alkyl;

Q and Z are independently selected from the group consisting of CH and N;

n is 0, 1, 2, 3 or 4;

s is 0, 1, 2, 3 or 4; and

t is 1, 2, 3 or 4;

with the proviso that when n is 0, Z is CH;

(ix) a compound represented by the structural Formula IX:

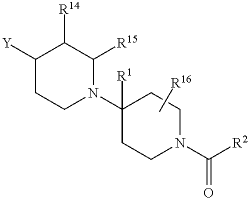

or a pharmaceutically acceptable salt, solvate or ester thereof; wherein:

Y is selected from the group consisting of R—X— and —N(R²⁰)(R²¹);

X is selected from the group consisting of —C(R¹³)₂—, —C(R¹³)(R¹⁹)—, —C(O)—, —O—,

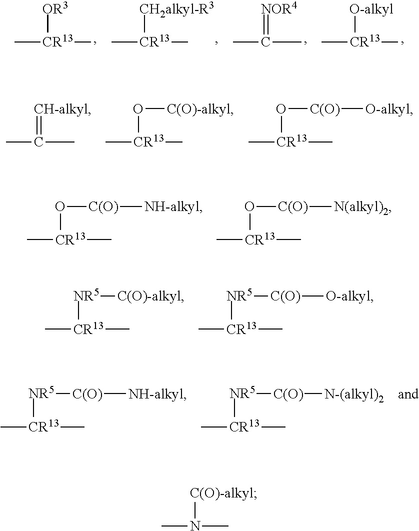

R is selected from the group consisting of R⁶-phenyl, R⁶-pyridyl, R⁶-thiophenyl, R⁶-naphthyl, and

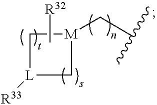

n is 0, 1, 2, 3 or 4;

s is 0, 1, 2, 3 or 4;

t is 1, 2, 3 or 4

L and M are independently selected from the group consisting of CH and N;

R¹ is selected from the group consisting of hydrogen, alkyl, and alkenyl;

R² is selected from the group consisting of

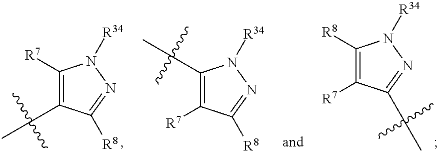

R³ is selected from the group consisting of R⁶-phenyl, R⁶-heteroaryl and R⁶-naphthyl;

R⁴ is selected from the group consisting of hydrogen, alkyl, fluoroalkyl, cyclopropylmethyl, —CH₂CH₂OH, —CH₂CH₂Oalkyl, —CH₂C(O)—O-alkyl, —CH₂C(O)NH₂, —CH₂C(O)—NHalkyl, and —CH₂C(O)—N(alkyl)₂;

R⁵ is selected from the group consisting of hydrogen and alkyl;

R⁶ is 1 to 3 substituents independently selected from the group consisting of hydrogen, halogen, alkyl, alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃)—, CH₃C(═NOCH₂CH₃)—,

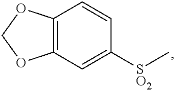

—NH₂, —NHCOCF₃, NHCONHalkyl, —NHC(O)alkyl, —NHS(O₂)alkyl, 5-membered heteroaryl and

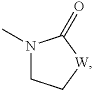

wherein W is —O—, —NH— or —N(CH₃)—;

each R⁷ and R⁸ is independently selected from the group consisting of hydrogen, alkyl, haloalkyl, halogen, —NR¹¹R¹², —OH, —CF₃, —O-alkyl, —O-acyl, and —O-haloalkyl;

R⁹ is selected from the group of consisting of hydrogen, alkyl, haloalkyl, cycloalkyl, aryl, acyl, heteroaryl, arylalkyl-, and heterocyclyl;

R¹¹, R¹², each R¹³, each R¹⁴, R¹⁵, R¹⁶, and R³² are independently selected from the group consisting of hydrogen and alkyl;

R¹⁹ is selected from the group consisting of R⁶-phenyl, R⁶-heteroaryl, R⁶-naphthyl, cycloalkyl, cycloalkylalkyl, and alkoxyalkyl;

R²⁰ is selected from the group consisting of hydrogen, alkyl, aryl, arylalkyl, heteroarylalkyl, alkyl-C(O)—, aryl-C(O)—, haloalkyl, cycloalkyl, heterocyclyl, cycloalkylalkyl, alkylsulfonyl, arylsulfonyl, alkoxysulfonyl,

alkoxyalkyl, and

### —N(R¹³)C(O)alkyl;

R²¹ is selected from the group consisting of:

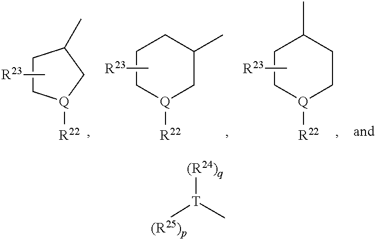

p is a number from 0-4;

q is a number from 0-4;

T is selected from the group consisting of aryl and heteroaryl, each of said aryl and heteroaryl being optionally independently substituted with R²⁴ and R²⁵;

R²² is selected from the group consisting of hydrogen, arylalkyl, alkyl, R²⁶-arylalkyl-, R²⁶-heteroarylalkyl-, alkylsulfonyl, cycloalkylsulfonyl, arylsulfonyl, R²⁶-arylsulfonyl-, —C(O)-alkyl, —C(O)-cycloalkyl, R²⁶-aryl-C(O)—, —C(O)NR²⁷R²⁸, and —SO₂NR²⁷R²⁸;

R²³ is selected from the group consisting of hydrogen, alkyl, and haloalkyl;

R²⁴ is selected from the group consisting of hydrogen, alkyl, haloalkyl, cycloalkyl, cycloalkylalkyl, heterocyclyl, aryl, arylalkyl, heteroarylalkyl, alkyl-C(O)—, aryl-C(O)—, alkylsulfonyl, arylsulfonyl, alkoxyalkyl, —N(R¹³)C(O)alkyl, and —C(O)N(R¹³)₂.

R²⁵ is selected from the group consisting of alkyl-C(O)-heterocyclyl, -alkyl-CN, -alkyl-N(R¹³)C(O)-alkyl-NR²⁹R³⁰, -alkyl-N(R¹³)C(O)-alkyl(aryl)-NR²⁹R³⁰, -alkyl-N(R¹³)C(O)-heterocyclyl, -alkyl-N(R¹³)C(O)-heteroalkyl, -alkyl-N(R¹³)C(O)-arylhydroxyalkyl, -alkyl-N(R¹³)C(O)—C(O)(aryl), -alkyl-N(R¹³)C(O)—C(O)alkyl, -alkyl-N(R¹³)C(O)—C(O)-heteroaryl, heterocyclyl, -alkyl-O—C(O)Z, -alkyl-S(O₂)-alkyl-NR²⁹R³⁰, -haloalkyl-C(O)OR³⁰, -haloalkyl-C(O)N(R³⁰R³¹), -alkyl-S(O₂)R³⁰, —S(O₂)-(hydroxyalkyl), -alkyl-C(O)R³⁰, -alkyl-C(R³⁰)(═N—OR³¹), —N(R¹³)C(O)-alkyl-N(CHR²⁹R³⁰), and —S(O₂)heterocyclyl;

Z is selected from the group consisting of heterocyclyl, NR³⁰R³¹, —O(alkyl), —O(cycloalkyl) and —OH;

R²⁶ is 1, 2, or 3 substituents independently selected from the group consisting of hydrogen, halo, alkyl, alkoxy, —CF₃, —OCF₃, CH₃C(O)—, —CN, CH₃SO₂—, CF₃SO₂—, and —NH₂;

R²⁷ is selected from the group consisting of H, alkyl and cycloalkyl;

R²⁸ is selected from the group consisting of alkyl, haloalkyl, hydroxyalkyl, cycloalkyl, aryl and arylalkyl;

or R²⁷ and R²⁸ together are (C₂-C₆) alkyl and form a ring with the nitrogen atom to which they are shown attached;

R²⁹ is selected from the group consisting of hydrogen, alkyl, cycloalkyl, heterocyclyl, hydroxyalkyl, aryl, heteroaryl, alkyloxy, alkylsulfonyl, cycloalkylsulfonyl, alkylarylsulfonyl, arylsulfonyl, C(O)alkyl, C(O)aryl, C(O)arylalkyl, C(O)cycloalkyl and —C(O)NR³⁰R³¹;

each R³⁰ and R³¹ are independently selected from the group consisting of hydrogen, alkyl, cycloalkyl, heterocyclyl, aryl, arylalkyl, and heteroaryl;

R³³ and R³⁶ are independently selected from the group consisting of hydrogen, -alkyl, fluoroalkyl-, R⁹-arylalkyl-, R⁹-heteroaryl-, alkylsulfonyl-, cycloalkylsulfonyl, fluoroalkylsulfonyl, R⁹-arylsulfonyl-, R⁹-heteroarylsulfonyl-, N(R²²)(R²³)—SO₂—, alkyl-C(O)—, cycloalkylC(O)—, fluoroalkyl-C(O)—, R⁹-aryl-C(O)—, NH-alkyl-C(O)— or R⁹-aryl-NH—C(O)—;

R³⁴ is selected from the group consisting of R³⁵O—, —NH₂, —NHR³⁵, and R³⁵R³⁶N—; and

each R³⁵ is independently selected from the group consisting of alkyl, haloalkyl, cycloalkyl, heterocyclyl, aralkyl, alkylaryl, aryl, and heteroaryl;

(x) a compound represented by the structural Formula X:

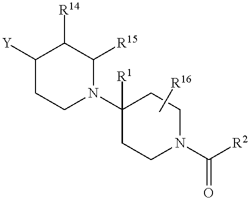

or a pharmaceutically acceptable salt, solvate or ester thereof, wherein:
Y is selected from the group consisting of R—X— and —N(R²⁰)(R²¹);
X is selected from the group consisting of —C(R¹³)₂—, —C(R¹³)(R¹⁹)—, —C(O), —O—,

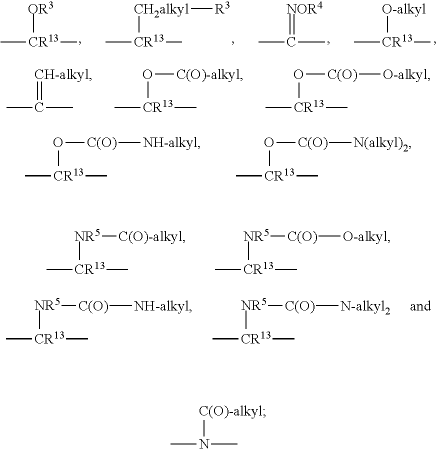

R is selected from the group consisting of R⁶-phenyl, R⁶-pyridyl, R⁶-thiophenyl, R⁶-naphthyl, and

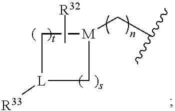

n is 0, 1, 2, 3 or 4;

s is 0, 1, 2, 3 or 4;

t is 1, 2, 3 or 4

L and M are independently selected from the group consisting of CH and N;

R¹ is selected from the group consisting of hydrogen, alkyl, and alkenyl;

R² is selected from the group consisting of

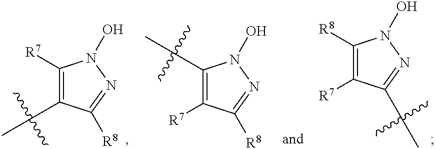

R³ is selected from the group consisting of R⁶-phenyl, R⁶-heteroaryl and R⁶-naphthyl;

R⁴ is selected from the group consisting of hydrogen, alkyl, fluoroalkyl, cyclopropylmethyl, —CH₂CH₂OH, —CH₂CH₂Oalkyl, —CH₂C(O)—O-alkyl, —CH₂C(O)NH₂, —CH₂C(O)—NHalkyl, and —CH₂C(O)—N(alkyl)₂;

R⁵ is selected from the group consisting of hydrogen and alkyl;

R⁶ is 1 to 3 substituents independently selected from the group consisting of hydrogen, halogen, alkyl, alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃)—, CH₃C(═NOCH₂CH₃)—,

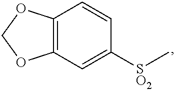

—NH₂, —NHCOCF₃, NHCONHalkyl, —NHC(O)alkyl, —NHS(O₂)alkyl, 5-membered heteroaryl and

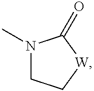

wherein W is —O—, —NH— or —N(CH₃)—;

each R⁷ and R⁸ are independently selected from the group consisting of hydrogen, alkyl, haloalkyl, halogen, —NR¹¹R¹², —OH, —CF₃, —O-alkyl, —O-acyl, and —O-haloalkyl;

R⁹ is selected from the group of consisting of hydrogen, alkyl, haloalkyl, cycloalkyl, aryl, acyl, heteroaryl, arylalkyl-, and heterocyclyl;

R¹¹, R¹², each R¹³, each R¹⁴, R¹⁵, R¹⁶, and R³² are independently selected from the group consisting of hydrogen and alkyl;

R¹⁹ is selected from the group consisting of R⁶-phenyl, R⁶-heteroaryl, R⁶-naphthyl, cycloalkyl, cycloalkylalkyl, and alkoxyalkyl;

R²⁰ is selected from the group consisting of hydrogen, alkyl, aryl, arylalkyl, heteroarylalkyl, alkyl-C(O)—, aryl-C(O)—, haloalkyl, cycloalkyl, heterocyclyl, cycloalkylalkyl, alkylsulfonyl, arylsulfonyl, alkoxysulfonyl, alkoxyalkyl, and

### —N(R¹³)C(O)alkyl;

R²¹ is selected from the group consisting of:

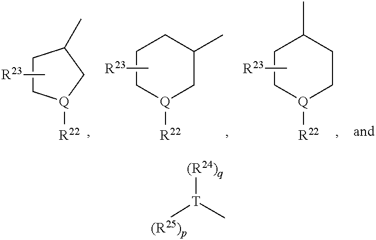

p is a number from 0-4;

q is a number from 0-4;

T is selected from the group consisting of aryl and heteroaryl, each of said aryl and heteroaryl being optionally independently substituted with R²⁴ and R²⁵;

R²² is selected from the group consisting of hydrogen, arylalkyl, alkyl, R²⁶-arylalkyl-, R²⁶-heteroarylalkyl-, alkylsulfonyl, cycloalkylsulfonyl, arylsulfonyl, R²⁶-arylsulfonyl-, —C(O)-alkyl, —C(O)-cycloalkyl, R²⁶-aryl-C(O)—, —C(O)NR²⁷R²⁸, and —SO₂NR²⁷R²⁸;

R²³ is selected from the group consisting of hydrogen, alkyl, and haloalkyl;

R²⁴ is selected from the group consisting of hydrogen, alkyl, haloalkyl, cycloalkyl, cycloalkylalkyl, heterocyclyl, aryl, arylalkyl, heteroarylalkyl, alkyl-C(O)—, aryl-C(O)—, alkylsulfonyl, arylsulfonyl, alkoxyalkyl, —N(R¹³)C(O)alkyl, and —C(O)N(R¹³)₂;

R²⁵ is selected from the group consisting of alkyl-C(O)-heterocyclyl, -alkyl-CN, -alkyl-N(R¹³)C(O)-alkyl-NR²⁹R³⁰, -alkyl-N(R¹³)C(O)-alkyl(aryl)-NR²⁹R³⁰, -alkyl-N(R¹³)C(O)-heterocyclyl, -alkyl-N(R¹³)C(O)-heteroalkyl, -alkyl-N(R¹³)C(O)— arylhydroxyalkyl, -alkyl-N(R¹³)C(O)—C(O)(aryl), -alkyl-N(R¹³)C(O)—C(O)alkyl, -alkyl-N(R¹³)C(O)—C(O)-heteroaryl, heterocyclyl, -alkyl-O—C(O)Z, -alkyl-S(O₂)-alkyl-NR²⁹R³⁰, -haloalkyl-C(O)OR³⁰, -haloalkyl-C(O)N(R³⁰R³¹), -alkyl-S(O₂)R³⁰, —S(O₂)— (hydroxyalkyl), -alkyl-C(O)R³⁰, -alkyl-C(R³⁰)(═N—OR³¹), —N(R¹³)C(O)-alkyl-N(CHR²⁹R³⁰), and —S(O₂)heterocyclyl;

Z is selected from the group consisting of heterocyclyl, NR³⁰R³¹, —O(alkyl), —O(cycloalkyl) and —OH;

R²⁶ is 1, 2, or 3 substituents independently selected from the group consisting of hydrogen, halo, alkyl, alkoxy, —CF₃, —OCF₃, CH₃C(O)—, —CN, CH₃SO₂—, CF₃SO₂—, and —NH₂;

R²⁷ is selected from the group consisting of H, alkyl and cycloalkyl;

R²⁸ is selected from the group consisting of alkyl, haloalkyl, hydroxyalkyl, cycloalkyl, aryl and arylalkyl;

or R²⁷ and R²⁸ together are (C₂-C₆) alkyl and form a ring with the nitrogen atom to which they are shown attached;

R²⁹ is selected from the group consisting of hydrogen, alkyl, cycloalkyl, heterocyclyl, hydroxyalkyl, aryl, heteroaryl, alkyloxy, alkylsulfonyl, cycloalkylsulfonyl, alkylarylsulfonyl, arylsulfonyl, C(O)alkyl, C(O)aryl, C(O)arylalkyl, C(O)cycloalkyl and —C(O)NR³⁰R³¹;

each R³⁰ and R³¹ are independently selected from the group consisting of hydrogen, alkyl, cycloalkyl, heterocyclyl, aryl, arylalkyl, and heteroaryl; and

R³³ is selected from the group consisting of hydrogen, -alkyl, fluoroalkyl-, R⁹-arylalkyl-, R⁹-heteroaryl-, alkylsulfonyl-, cycloalkylsulfonyl, fluoroalkylsulfonyl, R⁹-arylsulfonyl-, R⁹-heteroarylsulfonyl-, N(R²²)(R²³)—SO₂—, alkyl-C(O)—, cycloalkylC(O)—, fluoroalkyl-C(O)—, R⁹-aryl-C(O)—, alkyl-NH—C(O)— or R⁹-aryl-NH—C(O)—; or

(xi) a compound represented by the structural Formula XI:

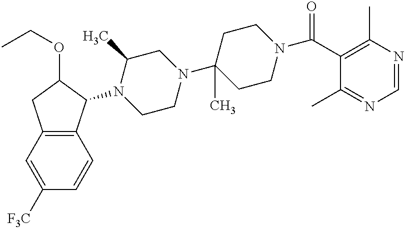

or a pharmaceutically acceptable salt or solvate thereof;

or a mixture of two or more compounds of Formula I to XI.

In some embodiments, the CCR5 antagonist or combination of CCR5 antagonists is administered via a vaginal device impregnated with the CCR5 antagonist, for example a vaginal ring device, an intrauterine device (IUD), vaginal diaphragm or vaginal sponge. The invention also encompasses a condom coated or impregnated with a CCR5 antagonist formulation.

In some embodiments, the present invention provides a topical cream, ointment or lotion formulation comprising:

(a) at least one compound of Formula Ito XI;
(b) at least one lubricant; and
(c) at least one adjuvant, wherein the adjuvant is an antimicrobial agent, antioxidant, humectant or emulsifier, or a mixture of two or more thereof.

The topical cream, ointment or lotion is suitable for vaginal, rectal or buccal administration.

In other embodiments, the present invention provides topical gel formulations comprising:

(a) at least one compound of Formula Ito XI;
(b) at least one antimicrobial agent; and
(c) at least one gelling agent.

In certain embodiments, the gel is suitable for vaginal, rectal or buccal administration.

In other embodiments, the present invention provides topical foam formulations comprising:

(a) at least one compound of Formula Ito XI;
(b) at least one antimicrobial agent;
© at least one emulsifier; and
(d) at least one propellant.

In certain embodiments, the foam is suitable for vaginal or rectal administration.

In other embodiments, the present invention provides vaginal or rectal suppositories, buccal or vaginal tablets, or buccal or Vaginal films.

In other embodiments, the present invention provides methods of preventing infection by HIV or inhibiting transmission of HIV comprising topically administering to a human in need of such prevention or at risk of such transmission an effective amount of any of the above formulations.

In other embodiments, the present invention provides methods of preventing infection by HIV or inhibiting transmission of HIV comprising administering to a human in need of such prevention or at risk of such transmission a CCR5 antagonist by inserting a vaginal device, preferably a vaginal ring device comprising or having impregnated therein or thereon a CCR5 antagonist.

In other embodiments, the present invention provides methods of inhibiting prophylactically an HIV infection of a subject by topical application of an antiviral effective amount of any of the above formulations. Other antiviral agents can be coadministered with the above formulations or devices.

In other embodiments, the present invention provides kits comprising in one or more separate or combined containers in a single package pharmaceutical compositions for use in combination to prevent infection by or inhibit transmission of Human Immunodeficiency Virus which Kits comprise in one container one of the above pharmaceutical formulations comprising a CCR5 antagonist, and in one or more separate containers, one or more pharmaceutical formulations comprising an effective amount of another antiviral or other agent useful in the prevention of Human Immunodeficiency Virus infection or transmission in a pharmaceutically acceptable carrier.

In certain specific embodiments of the CCR5 antagonist of the topical formulations or preparations (e.g., vaginal rings, etc.) of the present invention is the compound of structure

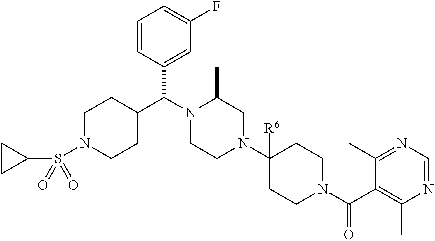

or the compound of structure

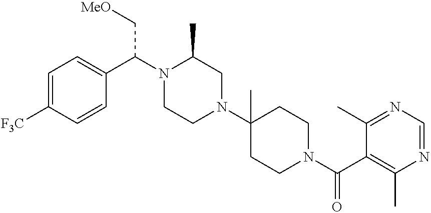

or a pharmaceutically acceptable salt of either of said compounds.

Other than in the operating examples, or where otherwise indicated, all numbers expressing quantities of ingredients, reaction conditions, and so forth used in the specification and claims are to be understood as being modified in all instances by the term “about.”

### BRIEF DESCRIPTION OF THE FIGURES

FIG. 1 illustrates PXRD of Forms 1 and 2 of Compound 5L.

FIG. 2 illustrates DSC of Forms 1 and 2 of Compound 5L.

FIG. 3 illustrates the thermogravimetric analysis (TGA) of Forms 1 and 2 of Compound 5L.

### DETAILED DESCRIPTION

The present invention provides topical preparations such as vaginal cream, vaginal ointment, vaginal lotion, vaginal gel, vaginal foam, vaginal suppository, vaginal tablet, vaginal film, rectal cream, rectal ointment, rectal lotion, rectal gel, rectal foam, rectal suppositories, buccal cream, buccal gel, buccal ointment, buccal lotion, buccal tablet or buccal film, etc.

The invention also provides at least one CCR5 antagonist in a form such as vaginal device such as vaginal rings, IUDs, sponges or diaphragms.

The topical preparations of the present invention can be used to prevent HIV infection in a human, or to inhibit transmission of the HIV virus from an infected human to another human. The topical preparations of the present invention can inhibit the growth or replication of a virus, such as a retrovirus, in particular a human immunodeficiency virus, specifically HIV-1 and HIV-2. The topical preparations are useful in the prophylactic treatment of humans who are at risk for viral infection. The topical preparations also can be used to treat objects or materials, such as contraceptive devices (for example condoms or intrauterine devices), medical equipment, supplies, or fluids, including biological fluids, such as blood, blood products, and tissues, to prevent or inhibit viral infection of a human. Such topical formulations also are useful to prevent sexual transmission of viral infections, e.g., HIV, which is the primary way in which HIV is transmitted globally.

The methods of prevention or inhibition or retardation of sexual transmission of viral infection, e.g., HIV infection, in accordance with the present invention, comprise vaginal, rectal, penile or other topical treatment with an antiviral effective amount of a topical preparation of the present invention, alone or in combination with another antiviral compound as described herein.

The topical preparations of the present invention comprise one or more of the compounds set forth above in Formulae Ito XI, which are CCR5 antagonists and are disclosed in U.S. Pat. No. 6,387,930 (Formulas I and II), U.S. Pat. No. 6,391,865 (Formulas III and IV), U.S. Pat. No. 6,720,325 (Formula V), U.S. Published Application 2007/0203149 (Formula VI), U.S. Published Application 2006/0223821 (Formulas VII and VIII), U.S. Published Application 2006/02238656 (Formulas IX and X), and U.S. Published Application 2005/0261310 (Formula XI) each incorporated by reference herein in their entirety. Methods of making such compounds are disclosed in the patents referenced above.

In some embodiments of compounds of formula I, R is R⁶-phenyl, especially wherein R⁶ is a single substituent, and especially wherein the R⁶ substituent is in the 4-position. In some embodiments, R¹³, R¹⁴, R¹⁵ and R¹⁶ are each hydrogen or methyl, especially hydrogen. Also preferred are compounds of formula I wherein X is —CHOR³, —C(R¹³)(R¹⁹)— or —C(═NOR⁴)—; a preferred definition for R³ is pyridyl, especially 2-pyridyl, a preferred definition for R⁴ is (C₁-C₆)alkyl, especially methyl, ethyl or isopropyl, a preferred definition for R¹³ is hydrogen, and a preferred definition for R¹⁹ is R⁶-phenyl. For compounds of formula I, R¹ is preferably (C₁-C₆)alkyl, especially methyl.

In compounds of formula I, R² is preferably R⁷, R⁸, R⁹-phenyl, R⁷, R⁸, R⁹-pyridyl or an N-oxide thereof, or R⁷, R⁸, R⁹-pyrimidyl. When R² is pyridyl, it is preferably 3- or 4-pyridyl, and when pyrimidyl, it is preferably 5-pyrimidyl. The R⁷ and R⁸ substituents are preferably attached to carbon ring members adjacent to the carbon joining the ring to the rest of the molecule and the R⁹ substituent can be attached to any of the remaining unsubstituted carbon ring members, for example as shown in the following structures:

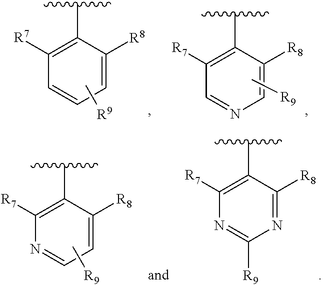

Preferred R7 and R8 substituents are: (C1-C6)alkyl, especially methyl; halogen, especially chloro; and —NH₂. A preferred R⁹ substituent is hydrogen.

Non-limiting examples of some embodiments of the compounds of Formula I include:

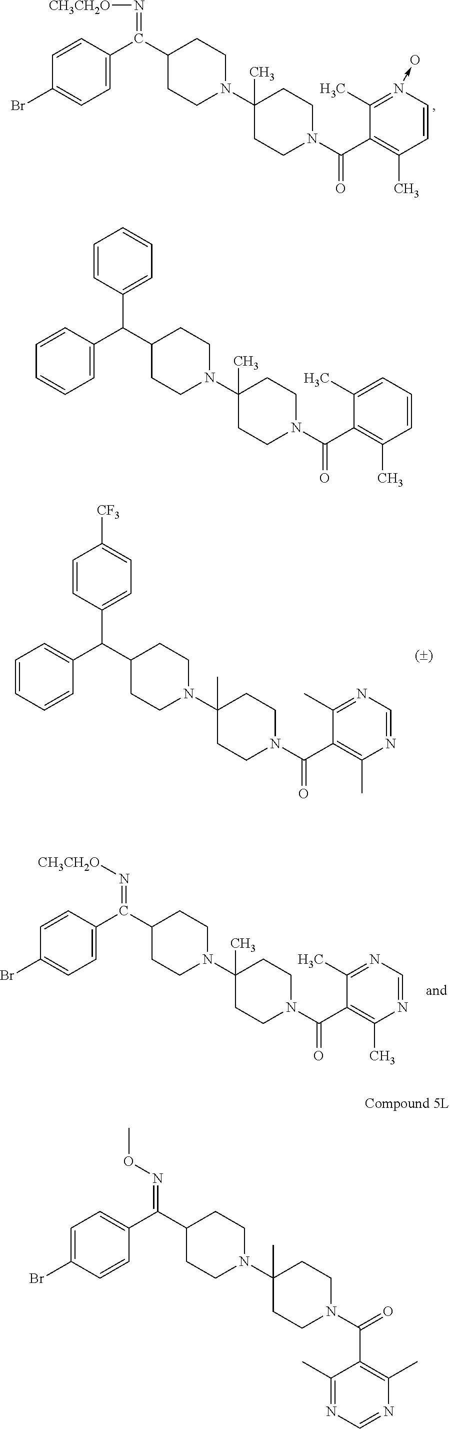

Compound 5L is described in Example 5 at cols. 53-69, see particularly col. 59 of U.S. Pat. No. 6,387,930, incorporated herein by reference.

In some embodiments of compounds of formula II(1), R^{a} is R^{6a}— phenyl, especially wherein R^{6a} is a single substituent, and especially wherein the R^{6a} substituent is in the 4-position. Also preferred are compounds of formula II(1) wherein X^{a} is —CHOR³, —C(R¹³)(R¹⁹)— or —C(═NOR⁴)—; a preferred definition for R³ is pyridyl, especially 2-pyridyl, a preferred definition for R⁴ is (C₁-C₆)alkyl, especially methyl, ethyl or isopropyl, a preferred definition for R¹³ is hydrogen, and a preferred definition for R¹⁹ is R⁶-phenyl. For compounds of formula II(1), R¹ is preferably (C₁-C₆)alkyl, especially methyl. Also for compounds of formula II(1), R¹⁴, R¹⁵ and R¹⁶ are preferably hydrogen.

Preferred are compounds of formula II(2) wherein R^{a} is R^{6b}-phenyl, especially wherein R^{6b} is a single substituent, and especially wherein the R^{6b} substituent is in the 4-position. Also preferred are compounds of formula II(2) wherein X^{a} is —CHOR³, —C(R¹³)(R¹⁹)— or —C(═NOR^{4a})—; a preferred definition for R³ is pyridyl, especially 2-pyridyl, preferred definitions for R^{4a} are cyclopropylmethyl and trifluoroethyl, a preferred definition for R¹³ is hydrogen, and a preferred definition for R¹⁹ is R⁶-phenyl. For compounds of formula II(2), R¹ is preferably (C₁-C₆)alkyl, especially methyl. Also for compounds of formula II(2), R¹⁴, R¹⁵ and R¹⁶ are preferably hydrogen.

In compounds of formula II(1) and (2), R² is preferably R⁷, R⁸, R⁹-phenyl; R⁷, R⁸, R⁹-pyridyl or an N-oxide thereof; or R⁷, R⁸, R⁹-pyrimidyl. When R² is pyridyl, it is preferably 3- or 4-pyridyl, and when pyrimidyl, it is preferably 5-pyrimidyl. The R⁷ and R⁸ substituents are preferably attached to carbon ring members adjacent to the carbon joining the ring to the rest of the molecule and the R⁹ substituent can be attached to any of the remaining unsubstituted carbon ring members as shown above for compounds of formula I. Preferred R⁷ and R⁸ substituents for compounds of formula II are: (C₁-C₆)alkyl, especially methyl; halogen, especially chloro; and —NH₂; a preferred R⁹ substituent is hydrogen.

In some embodiments of compounds of formula III, R is R⁸-phenyl or R⁸-naphthyl, especially wherein R⁸ is a single substituent, and especially wherein the R⁸ substituent is in the 4-position. For R⁸-phenyl, preferred R⁸ substituents are —CF₃, —OCF₃, CH₃SO₂—, CH₃CO—, CH₃C(═NOCH₃)—, Br and I. For R⁸-naphthyl, R⁸ is preferably C₁-C₆ alkoxy. Also preferred are compounds of formula III wherein R³ is hydrogen, (C₁-C₆)alkyl, R⁸-phenyl. R⁸-benzyl or R⁸-pyridyl; more preferred definitions for R³ are methyl, ethyl, phenyl, benzyl and pyridyl. R¹ is preferably hydrogen. For compounds of formula III, R⁶ is preferably hydrogen or methyl, especially methyl. R⁴ is preferably methyl; R⁵ and R⁷ are each preferably hydrogen.

In compounds of formula III, R² is preferably R⁹, R¹⁰, R¹¹-phenyl, R⁹, R¹⁰, R¹¹-pyridyl or an N-oxide thereof, or R⁹, R¹⁰, R¹¹-pyrimidyl. When R² is pyridyl, it is preferably 3- or 4-pyridyl, and when pyrimidyl, it is preferably 5-pyrimidyl. The R⁹ and R¹⁰ substituents are preferably attached to carbon ring members adjacent to the carbon joining the ring to the rest of the molecule and the R¹¹ substituent can be attached to any of the remaining unsubstituted carbon ring members, for example as shown in the following structures:

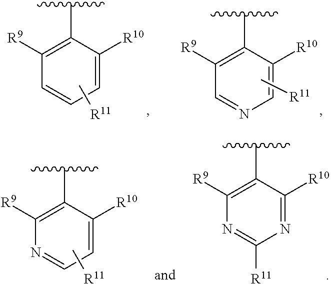

Preferred R⁹ and R¹⁰ substituents are: (C₁-C₆)alkyl, especially methyl; halogen, especially chloro or bromo, —OH and —NH₂. When R² is phenyl, R¹¹ is preferably hydrogen or —OH; when R² is pyridyl, R¹¹ is preferably hydrogen; and when R² is pyrimidyl, R¹¹ is preferably hydrogen, methyl or phenyl. Examples of particularly preferred R² groups are as follows:

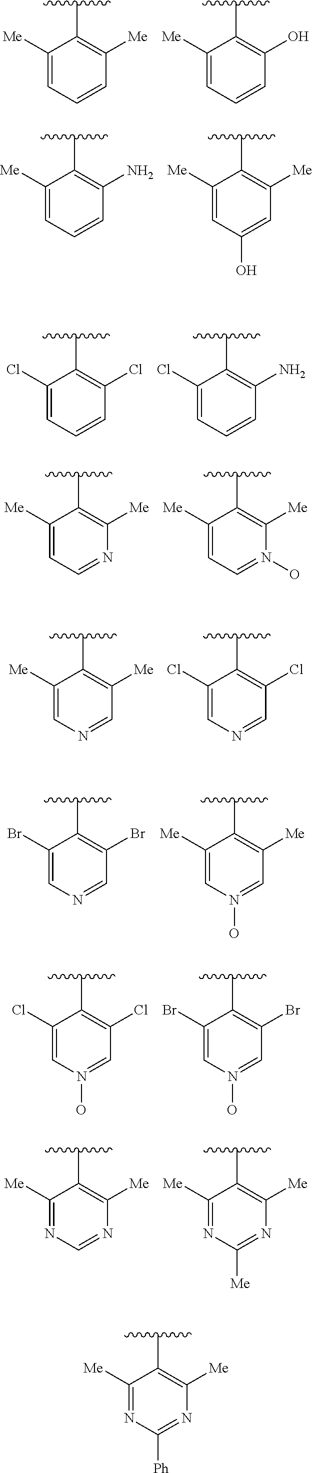

Non-limiting examples of compounds of Formula III are

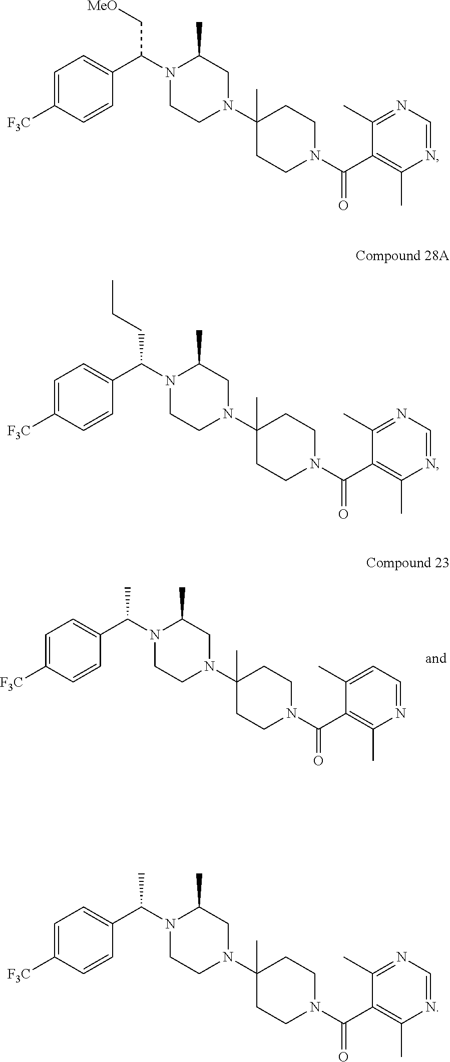

Compound 28A is described in Example 28 at cols 109-111 of U.S. Pat. No. 6,391,865, incorporated herein by reference. Compound 23 and hydrochloride salt is described at cols. 85-87, line 12, Example 23 of U.S. Pat. No. 6,391,865, incorporated herein by reference.

The compound of Formula IIIa is known as Vicriviroc:

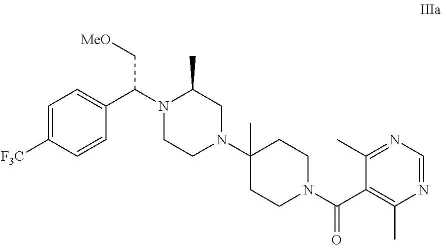

described in Example 29 at cols. 114-116, especially compound 29A of U.S. Pat. No. 6,391,865, incorporated herein by reference.

In some embodiments, compounds of Formula IV are those wherein R^{a} is R^{8a}-phenyl or R⁸-naphthyl, wherein R^{8a} is —CF₃, CF₃O— or halogen and R⁸ is C₁-C₆ alkoxy. The R^{8a} or R⁸ substituent is preferably a single substituent; it is especially preferred that the R^{8a} or R⁸ substituent is in the 4-position. Also preferred are compounds of formula IV (1) wherein R³ is hydrogen, (C₁-C₆)alkyl, R⁸-phenyl. R⁸-benzyl or R⁸-pyridyl; more preferred definitions for R³ are methyl, ethyl, phenyl, benzyl and pyridyl. R¹ is preferably hydrogen. For compounds of formula IV(1), R⁶ is preferably hydrogen or methyl, especially methyl. R⁴ is preferably methyl; R⁵ and R⁷ are each preferably hydrogen.

R² in formula IV(1) is preferably as defined for formula I, i.e., R⁹, R¹⁰, R¹¹-phenyl, R⁹, R¹⁰, R¹¹-pyridyl or an N-oxide thereof, or R⁹, R¹⁰, R¹¹-pyrimidyl, wherein the R⁹, R¹⁰, R¹¹-substitution is as defined above for preferred compounds of formula III.

In some embodiments, compounds of formula V include those wherein Z is CH, and Q and X are each N. Also preferred are compounds of formula V wherein R¹ is R⁹-aryl(C₁-C₆)alkyl-, R⁹-heteroaryl(C₁-C₆)alkyl-, (C₁-C₆)alkyl-SO₂—, (C₃-C₆)cycloalkyl-SO₂—, fluoro-(C₁-C₆)-alkyl-SO₂—, R⁹-aryl-SO₂—, or R⁹-aryl-NH—C(O)—. More preferably, R¹ is (C₁-C₆)alkyl-SO₂—, (C₃-C₆)cycloalkyl-SO₂— or R⁹-aryl-SO₂—. Preferably R² is hydrogen and R³ is (C₁-C₆)alkyl, R⁹-aryl, R⁹-aryl(C₁-C₆)-alkyl, R⁹-heteroaryl, or R⁹-heteroaryl(C₁-C₆)alkyl. When R² comprises an arylalkyl or heteroarylalkyl group, the alkyl portion of the arylalkyl or heteroarylalkyl preferably is methyl. R, R⁵ and R⁷ are preferably hydrogen. R⁴ is preferably (C₁-C₆)alkyl, more preferably methyl, when X is N; R⁴ is preferably H when X is CH. R⁶ is preferably —CH₃. R⁹ is preferably H, halogen, (C₁-C₆) alkyl or (C₁-C₆) alkoxy. When R¹ or R³ comprises an aryl or heteroaryl group, a preferred aryl group is phenyl, and preferred heteroaryl groups are thienyl, pyridyl and pyrimidyl.

In compounds of formula V, R⁸ is preferably (R¹⁴, R¹⁵, R¹⁶)-phenyl; (R¹⁴, R¹⁵, R¹⁶)-pyridyl or an N-oxide thereof; or (R¹⁴, R¹⁵, R¹⁶)-pyrimidyl. When R⁸ is pyridyl, it is preferably 3- or 4-pyridyl, and when pyrimidyl, it is preferably 5-pyrimidyl. The R¹⁴ and R¹⁵ substituents are preferably attached to carbon ring members adjacent to the carbon joining the ring to the rest of the molecule and the R¹⁶ substituent can be attached to any of the remaining unsubstituted carbon ring members. Thus, structures of the preferred R⁸ substituents are shown as follows:

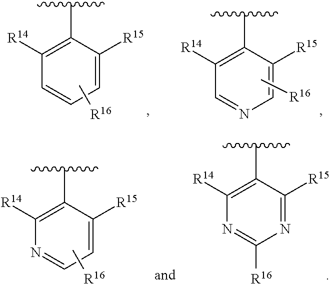

Preferred R¹⁴ and R¹⁵ substituents for compounds of formula V are: (C₁-C₆)alkyl, especially methyl; halogen, especially chloro; and —NH₂; a preferred R¹⁶ substituent is hydrogen.

In some non-limiting embodiments, the compound of Formula V is

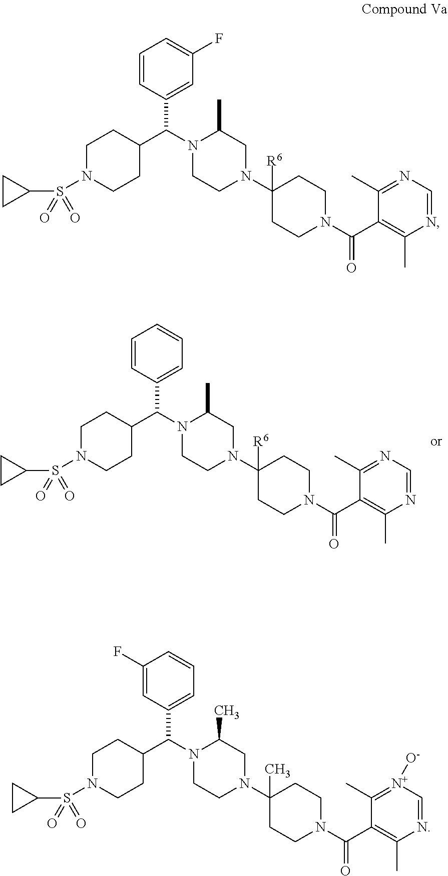

In some non-limiting embodiments of structural Formula VII or VIII, R¹ is R⁹-phenyl.

In another embodiment for the compounds of structural Formula VII or VIII, R¹ is

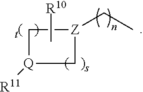

In another embodiment, for structural Formula VII or VIII, wherein R¹ is

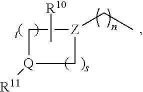

### Z is CH, and Q is N.

In another embodiment, for structural Formula VII or VIII, R² is hydrogen and R³ is selected from the group consisting of (C₁-C₆)alkyl, (C₁-C₆)alkoxy(C₁-C₆)alkyl-, and R⁹-aryl.

In a non-limiting embodiment of the compounds of Formula IX and X, R¹ is R⁹-phenyl.

In another embodiment of the compounds of Formula IX and X, R is

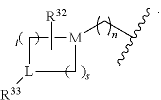

In another embodiment, wherein for Formula IX and X, wherein R is

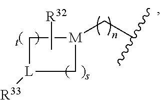

### M is CH and L is N.

In another embodiment of the compounds of Formula IX and Formula X, X is selected from the group consisting of —C(R¹³)₂— and —C(R¹³)(R¹⁹)—.

In another embodiment of the compounds of Formula IX and Formula X, X is —C(R¹³)₂—.

In another embodiment of the compounds of Formula IXand X, X is —C(R¹³)(R¹⁹)—.

As used herein, the following terms are as defined below unless otherwise indicated:

“Active compound” means a CCR5 receptor antagonist.

“At least one” CCR5 receptor antagonist means 1-3, preferably 1-2, more preferably 1 CCR5 receptor antagonist can be present. For the remaining optional components of the various formulations (e.g., the lubricant, emulsifier) the term “at least one” means 1-5.

Alkyl (including the alkyl portions of alkoxy, alkylamino and dialkylamino) represents straight and branched carbon chains and contains from one to six carbon atoms.

Fluoroalkyl represents an alkyl group as defined substituted by one or more fluorine atoms. Examples are —CH₂F, —CHF₂, —CF₃, —CH₂CF₃, —CF₂CF₃ and the like.

Hydroxyalkyl represents an alkyl group as defined substituted by 1 to 3 hydroxy groups.

Alkenyl represents C₂-C₆ carbon chains having one or two unsaturated bonds, provided that two unsaturated bonds are not adjacent to each other.

Substituted phenyl means that the phenyl group can be substituted at any available position on the phenyl ring.

Acyl means a radical of a carboxylic acid having the formula alkyl-C(O)—, aryl-C(O)—, aralkyl-C(O)—, (C₃-C₇)cycloalkyl-C(O)—, (C₃-C₇)cycloalkyl-(C₁-C₆)alkyl-C(O)—, and heteroaryl-C(O)—, wherein alkyl and heteroaryl are as defined herein.

Aryl is phenyl or naphthyl.

Heteroaryl represents cyclic aromatic groups of 5 or 6 atoms or bicyclic groups of 11 to 12 atoms having 1 or 2 heteroatoms independently selected from O, S or N, said heteroatom(s) interrupting a carbocyclic ring structure and having a sufficient number of delocalized pi electrons to provide aromatic character, provided that the rings do not contain adjacent oxygen and/or sulfur atoms. Nitrogen atoms can form an N-oxide. For 6-membered heteroaryl rings at R⁸, available carbon atoms can be substituted by R¹⁴, R¹⁵ or R¹⁶ groups. All regioisomers are contemplated, e.g., 2-pyridyl, 3-pyridyl and 4-pyridyl. Typical 6-membered heteroaryl groups are pyridyl, pyrimidinyl, pyrazinyl, pyridazinyl and the N-oxides thereof. For 5-membered heteroaryl rings at R⁸, available carbon atoms can be substituted by R¹⁷ or R¹⁸ groups. R⁹-substituted heteroaryl rings can be substituted on available carbon atoms by 1, 2 or 3 independently selected R⁹ groups. Typical 5-membered heteroaryl rings are furyl, thienyl, pyrrolyl, thiazolyl, isothiazolyl, imidazolyl, pyrazolyl and isoxazolyl. 5-Membered rings having one heteroatom can be joined through the 2- or 3-position; 5-membered rings having two heteroatoms are preferably joined through the 4-position. Bicyclic groups typically are benzo-fused ring systems derived from the heteroaryl groups named above, e.g. quinolyl, phthalazinyl, quinazolinyl, benzofuranyl, benzothienyl and indolyl.

Halogen represents fluoro, chloro, bromo and iodo.

As used herein, the terms “CCR5 antagonist” and CCR5 receptor antagonist” are interchangeable.

Certain CCR5 antagonist compounds suitable for the formulations and methods of the invention may exist in different isomeric forms (e.g., enantiomers, diastereoisomers and atropisomers). The invention encompasses all such isomers both in pure form and in admixture, including racemic mixtures.

Certain compounds suitable for the formulations or preparations and methods of the invention will be acidic in nature, e.g. those compounds which possess a carboxyl or phenolic hydroxyl group. These compounds may form pharmaceutically acceptable salts which are also suitable for the present invention. Examples of such salts may include sodium, potassium, calcium, aluminum, gold and silver salts. Also encompassed are salts formed with pharmaceutically acceptable amines such as ammonia, alkyl amines, hydroxyalkylamines, N-methylglucamine and the like.

Certain basic compounds suitable for the formulations or preparations and methods of the invention also form pharmaceutically acceptable salts, e.g., acid addition salts. For example, the pyrido-nitrogen atoms may form salts with strong acid, while compounds having basic substituents such as amino groups also form salts with weaker acids. Examples of suitable acids for forming a salt suitable for the present invention are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salts are prepared by contacting the free base form with a sufficient amount of the desired acid to produce a salt in the conventional manner. The free base forms may be regenerated by treating the salt with a suitable dilute aqueous base solution such as dilute aqueous NaOH, potassium carbonate, ammonia and sodium bicarbonate. The free base forms differ from their respective salt forms somewhat in certain physical properties, such as solubility in polar solvents, but the acid and base salts are otherwise equivalent to their respective free base forms for purposes of the invention.

For certain embodiments, for example vaginal ring formulations, the free base of a CCR5 antagonist is preferred.

Preferably, the pharmaceutical formulation is in a unit dosage form. In such form, the preparation is subdivided into suitably sized unit doses containing appropriate quantities of the active component, e.g., an effective amount to achieve the desired purpose.

The actual dosage of the active compound employed may be varied depending upon the requirements of the patient and the type of dosage form. For example, the dosage amount of a CCR5 antagonist present in a topical formulation that may be applied frequently but which does not remain in contact with the patient for prolonged periods of time may be lower than the dosage level in a slow-release vaginal ring device. Determination of the proper dosage regimen for a particular situation is within the skill of the art.

The amount and frequency of administration of the active compound employed and/or the pharmaceutically acceptable salts thereof will be regulated according to the judgment of the attending clinician considering such factors as age, condition and size of the patient. A typical recommended dosage regimen can range from about 10 mg/dose to about 100 mg/dose, preferably about 10 to about 50 mg/dose, and more preferably about 20 to about 25 mg/dose; when administered from a controlled-release device such as a vaginal ring device, the release of the CCR5 antagonist should be at a rate of about 10 to about 100 mg per day.

In the cream or ointment embodiments of the present invention, the topical formulation comprises one or more lubricants. The gels and foams of the present invention optionally can include one or more lubricants.

Non-limiting examples of useful lubricants include cetyl esters wax, hydrogenated vegetable oil, magnesium stearate, methyl stearate, mineral oil, polyoxyethylene-polyoxypropylene copolymer, polyethylene glycol, polyvinyl alcohol, sodium lauryl sulfate, white wax, or mixtures of two or more of the above.

The amount of lubricant in the topical formulation can range from about 0 to about 95 weight percent. Typical cream and ointment formulations comprise 0.1 to 95 weight percent of lubricant.

The topical formulations can comprise one or more adjuvants, wherein the adjuvant is an antimicrobial agent, antioxidant, humectant or emulsifier, or mixture of two or more thereof. The gels and foams of the present invention can include one or more antimicrobial agents and optionally can include one or more of antioxidants, humectants and emulsifiers.

Non-limiting examples of useful antimicrobial agents are benzyl alcohol, propylene glycol, propyl paraben, methyl paraben, or mixtures of two or more thereof.

The amount of antimicrobial agents in the topical formulation can range from about 0.01 to about 10 weight percent, and in some embodiments from about 0.2 to about 10 weight percent, on a basis of total weight of the topical formulation.

Non-limiting examples of useful antioxidants include butylated hydroxyanisole, butylated hydroxytoluene, edetate disodium or mixtures of two or more thereof.

The amount of antioxidant in the topical formulation can range from about 0.01 to about 1 weight percent, and in some embodiments from about 0.01 to about 0.1 weight percent, on a basis of total weight of the topical formulation.

Non-limiting examples of useful humectants include ethylene glycol, glycerin, sorbitol or mixtures of two or more thereof.

The amount of humectant in the topical formulation can range from about 1 to about 30 weight percent, and in some embodiments from about 2 to about 20 weight percent, on a basis of total weight of the topical formulation.

Non-limiting examples of useful emulsifiers include carbomers (such as Carbomer 934P, manufactured by Voveon, inc.), polyoxyethylene-10-stearyl ether, polyoxyethylene-20-stearyl ether, cetostearyl alcohol, cetyl alcohol, cholesterol, diglycol stearate, glyceryl monostearate, glyceryl stearate, polyglyceryl-3-oleate, hydroxypropyl cellulose, hydroxypropylmethyl cellulose, lanolin, polyoxyethylene lauryl ether, methyl cellulose, polyoxyethylene stearate, polysorbate, propylene glycol monostearate, sorbitan esters, stearic acid or mixtures of two or more thereof.

The amount of emulsifier in the topical formulation can range from about 1 to about 40 weight percent, and in some embodiments from about 5 to about 30 weight percent, on a basis of total weight of the topical formulation.

The gel formulations of the present invention comprise one or more gelling agents. Non-limiting examples of useful gelling agents include carbomer, cetostearyl alcohol, hydroxymethyl cellulose, polyoxyethylene-polyoxypropylene copolymer, sodium carboxymethylcellulose, or mixtures of two or more thereof.

The amount of gelling agent in the topical gel formulation can range from about 0.1 to about 10 weight percent, and in some embodiments from about 0.1 to about 1 weight percent, on a basis of total weight of the topical formulation.

The gel formulations of the present invention can further comprise one or more alkalinizers, for example sodium hydroxide, in amount of less than about 2 weight percent.

The formulations can contain one or more additional excipients well known in the art, for example water and a thickening agent such as colloidal silicon dioxide.

The formulations of the present invention can be administered in combination with one or more other antiviral or other agents useful in treating or preventing infection with HIV or in inhibiting transmission of HIV, in combination with a pharmaceutically acceptable carrier.

One or more, preferably one to four, antiviral agents useful in anti-HIV-1 therapy may be used in combination with at least one (i.e., 1-4, preferably 1) CCR5 antagonist in a formulation of the present invention. The antiviral agent or agents may be combined with the CCR5 antagonist in a single dosage form, or the CCR5 antagonist and the antiviral agent or agents may be administered simultaneously or sequentially as separate dosage forms. For example, the CCR5 formulation can be used in a vaginal ring device or to coat the outside of a condom to prevent transmission of HIV to a non-infected sexual partner while the HIV-infected sexual partner undergoes treatment with systemic antiviral therapy. The antiviral agents contemplated for use in combination with the CCR5 antagonist formulations of the present invention comprise nucleoside and nucleotide reverse transcriptase inhibitors, non-nucleoside reverse transcriptase inhibitors, protease inhibitors and other antiviral drugs listed below not falling within these classifications. In particular, the combinations known as HAART are contemplated for use in combination with the CCR5 antagonist formulations of this invention.

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA.

Typical suitable NRTIs include zidovudine (AZT) available under the RETROVIR tradename from Glaxo-Wellcome Inc., Research Triangle, NC 27709; didanosine (ddI) available under the VIDEX tradename from Bristol-Myers Squibb Co., Princeton, N.J. 08543; zalcitabine (ddC) available under the HIVID tradename from Roche Pharmaceuticals, Nutley, N.J. 07110; stavudine (d4T) available under the ZERIT trademark from Bristol-Myers Squibb Co., Princeton, N.J. 08543; lamivudine (3TC) available under the EPIVIR tradename from Glaxo-Smith Kline Triangle, NC 27709; abacavir (1592U89) disclosed in WO96/30025 and available under the ZIAGEN trademark from Glaxo-Wellcome Research Triangle, NC 27709; adefovir dipivoxil [bis(POM)-PMEA] available under the PREVON tradename from Gilead Sciences, Foster City, Calif. 94404; lobucavir (BMS-180194), a nucleoside reverse transcriptase inhibitor disclosed in EP-0358154 and EP-0736533 and under development by Bristol-Myers Squibb, Princeton, N.J. 08543; BCH-10652, a reverse transcriptase inhibitor (in the form of a racemic mixture of BCH-10618 and BCH-10619) under development by Biochem Pharma, Laval, Quebec H7V, 4A7, Canada; emitricitabine[(−)-FTC] licensed from Emory University under Emory Univ. U.S. Pat. No. 5,814,639 and available from Gilead under the trade name Emtrivia™; beta-L-FD4 (also called beta-L-D4C and named beta-L-2′,3′-dicleoxy-5-fluoro-cytidene) licensed by Yale University to Vion Pharmaceuticals, New Haven Conn. 06511; DAPD, the purine nucleoside, (−)-beta-D-2,6,-diamino-purine dioxolane disclosed in EP 0656778 and licensed by Emory University and the University of Georgia to Triangle Pharmaceuticals, Durham, N.C. 27707; and lodenosine (FddA), 9-(2,3-dideoxy-2-fluoro-b-D-threo-pentofuranosyl)adenine, an acid stable purine-based reverse transcriptase inhibitor discovered by the NIH and under development by U.S. Bioscience Inc., West Conshohoken, Pa. 19428.

The term “non-nucleoside reverse transcriptase inhibitors” (“NNRTI”s) as used herein means non-nucleosides that inhibit the activity of HIV-1 reverse transcriptase.

Typical suitable NNRTIs include nevirapine (BI-RG-587) available under the VIRAMUNE tradename from Boehringer Ingelheim, the manufacturer for Roxane Laboratories, Columbus, Ohio 43216; delaviradine (BHAP, U-90152) available under the RESCRIPTOR tradename from Pharmacia & Upjohn Co., Bridgewater N.J. 08807; efavirenz (DMP-266) a benzoxazin-2-one disclosed in WO94/03440 and available under the SUSTIVA tradename from Bristol Myers Squibb in the US and Merck in Europe; PNU-142721, a furopyridine-thio-pyrimide under development by Pharmacia and Upjohn, Bridgewater N.J. 08807; AG-1549 (formerly Shionogi # S-1153); 5-(3,5-dichlorophenyl)-thio-4-isopropyl-1-(4-pyridyl)methyl-IH-imidazol-2-ylmethyl carbonate disclosed in WO 96/10019 and under clinical development by Agouron Pharmaceuticals, Inc., LaJolla Calif. 92037-1020; MKC-442 (1-(ethoxy-methyl)-5-(1-methylethyl)-6-(phenylmethyl)-(2,4(1H,3H)-pyrimidinedione) discovered by Mitsubishi Chemical Co. and under development by Triangle Pharmaceuticals, Durham, N.C. 27707; (+)-calanolide A (NSC-675451) and B, coumarin derivatives disclosed in NIH U.S. Pat. No. 5,489,697, licensed to Med Chem Research, which is co-developing (+) calanolide A with Vita-Invest as an orally administrable product; and etravirine (TMC-125, Intelence) marketed by Tibotec.

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character, e.g. CRIXIVAN (available from Merck) as well as nonpeptide protease inhibitors e.g., VIRACEPT (available from Agouron).

Typical suitable PIs include saquinavir (Ro 31-8959) available in hard gel capsules under the INVIRASE tradename and as soft gel capsules under the FORTOVASE tradename from Roche Pharmaceuticals, Nutley, N.J. 07110-1199; ritonavir (ABT-538) available under the NORVIR tradename from Abbott Laboratories, Abbott Park, Ill. 60064; indinavir (MK-639) available under the CRIXIVAN tradename from Merck & Co., Inc., West Point, Pa. 19486-0004; nelfnavir (AG-1343) available under the VIRACEPT tradename from Agouron Pharmaceuticals, Inc., LaJolla Calif. 92037-1020; amprenavir (141W94), tradename AGENERASE, a non-peptide protease inhibitor under development by Vertex Pharmaceuticals, Inc., Cambridge, Mass. 02139-4211 and available from Glaxo-Wellcome, Research Triangle, NC under an expanded access program; lasinavir (BMS-234475) available from Bristol-Myers Squibb, Princeton, N.J. 08543 (originally discovered by Novartis, Basel, Switzerland (CGP-61755); DMP-450, a cyclic urea discovered by Dupont and under development by Triangle Pharmaceuticals; BMS-2322623, an azapeptide under development by Bristol-Myers Squibb, Princeton, N.J. 08543, as a 2nd-generation HIV-1 PI; ABT-378 under development by Abbott, Abbott Park, Ill. 60064; AG-1549 an orally active imidazole carbamate discovered by Shionogi (Shionogi #5-1153) and under development by Agouron Pharmaceuticals, Inc., LaJolla Calif. 92037-1020; atazanavir; tipranavir; and darunavir.

Other antiviral agents include CXCR4 antagonists, enfuvirtide, hydroxyurea, ribavirin, IL-2, IL-12, pentafuside and Yissum Project No. 11607. Hydroxyurea (Droxia), a ribonucleoside triphosphate reductase inhibitor, the enzyme involved in the activation of T-cells, was discovered at the NCI and is under development by Bristol-Myers Squibb; in preclinical studies, it was shown to have a synergistic effect on the activity of didanosine and has been studied with stavudine. IL-2 is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314, and is available under the PROLEUKIN (aldesleukin) tradename from Chiron Corp., Emeryville, Calif. 94608-2997 as a lyophilized powder for IV infusion or sc administration upon reconstitution and dilution with water; a dose of about 1 to about 20 million IU/day, sc is preferred; a dose of about 15 million IU/day, sc is more preferred. IL-12 is disclosed in WO96/25171 and is available from Roche Pharmaceuticals, Nutley, N.J. 07110-1199 and American Home Products, Madison, N.J. 07940; a dose of about 0.5 microgram/kg/day to about 10 microgram/kg/day, sc is preferred. Enfuvirtide (DP-178, T-20) a 36-amino acid synthetic peptide, is disclosed in U.S. Pat. No. 5,464,933 licensed from Duke University to Trimeris which developed enfuvirtide in collaboration with Duke University and Roche; enfuvirtide acts by inhibiting fusion of HIV-1 to target membranes. Enfuvirtide (3-100 mg/day) is given as a continuous sc infusion or injection together with efavirenz and 2 PI's to HIV-1 positive patients refractory to a triple combination therapy; use of 100 mg/day is preferred. Yissum Project No. 11607, a synthetic protein based on the HIV-1 Vif protein, is under preclinical development by Yissum Research Development Co., Jerusalem 91042, Israel. Ribavirin, 1-β-D-ribofuranosyl-1H-1,2,4-triazole-3-carboxamide, is available from ICN Pharmaceuticals, Inc., Costa Mesa, Calif.; its manufacture and formulation are described in U.S. Pat. No. 4,211,771; the integrase inhibitor raltegravir available from Merck under the tradename Isentress™; elvitegravir an integrase inhibitor under development by Gilead Sciences; the HIV-1Gag maturation inhibitor berivimat under development (Phase IIb) by Panacos Pharmaceuticals.

The term “anti-HIV-1 therapy” as used herein means any anti-HIV-1 drug found useful for treating HIV-1 infections in man alone, or as part of multidrug combination therapies, especially the HAART triple and quadruple combination therapies. Typical suitable known anti-HIV-1 therapies include, but are not limited to multidrug combination therapies such as (i) at least three anti-HIV-1 drugs selected from two NRTIs, one PI, a second PI, and one NNRTI; and (ii) at least two anti-HIV-1 drugs selected from NNRTIs and PIs. Typical suitable HAART-multidrug combination therapies include:

(a) triple combination therapies such as two NRTIs and one PI; or (b) two NRTIs and one NNRTI; and (c) quadruple combination therapies such as two NRTIs, one PI and a second PI or one NNRTI. In treatment of naive patients, it is preferred to start anti-HIV-1 treatment with the triple combination therapy; the use of two NRTIs and one NNRTI or two NRTIs and one PI is preferred if there is intolerance to NNRTI. Drug compliance is essential. The CD4⁺ and HIV-1-RNA plasma levels should be monitored every 3-6 months. Should viral load plateau, a fourth drug, e.g., one PI, one NNRTI or integrase inhibitor could be added. See the table below wherein typical therapies are further described:

### Anti-HIV-1 Multi Drug Combination Therapies

### A. Triple Combination Therapies

### 1. Two NRTIs¹+one PI²

### 2. Two NRTIs¹+one NNRTI³

### B. Quadruple Combination Therapies⁴

Two NRTIs+one PI+a second PI or one NNRTI

### C. Alternatives:⁵

Two NRTI¹

One NRTI⁵+one PI²

Two PIs⁶±one NRTI⁷ or NNRTI³

One PI²+one NRTI⁷+one NNRTI³

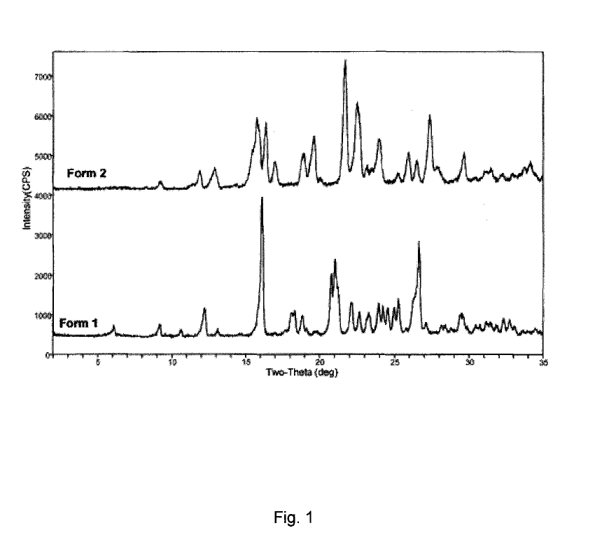

Footnotes to Table 1. One of the following: zidovudine+lamivudine; zidovudine+didanosine; stavudine+lamivudine; stavudine+didanosine; zidovudine+zalcitabine2. Indinavir, nelfinavir, ritonavir or saquinavir soft gel capsules.3. Nevirapine or delavirdine.4. See A-M. Vandamne et al Antiviral Chemistry & Chemotherapy 9:187 at p 193-197 and FIGS. 1+2.5. Alternative regimens are for patients unable to take a recommended regimen because of compliance problems or toxicity, and for those who fail or relapse on a recommended regimen. Double nucleoside combinations may lead to HIV-resistance and clinical failure in many patients.6. Most data obtained with saquinavir and ritonavir (each 400 mg bid).7. Zidovudine, stavudine or didanosine.

The doses and dosage regimens of the NRTIs, NNRTIs, PIs and other agents used in combination with the CCR5 antagonist formulation will be determined by the attending clinician in view of the approved doses and dosage regimens in the package inserts or as set forth in the protocols, taking into consideration the age, sex and condition of the patient and the severity of the condition treated.

In certain embodiments of the present invention, the goal of the formulations of the present invention is to reduce the HIV-1-RNA viral load below the detectable limit so that infection or transmission of infection is slowed, prevented or inhibited. The “detectable limit of HIV-1-RNA” in the context of the present invention means that there are fewer than about 200 to fewer than about 50 copies of HIV-1-RNA per ml of plasma of the patient as measured by quantitative, multi-cycle reverse transcriptase PCR methodology. HIV-1-RNA is preferably measured in the present invention by the methodology of Amplicor-1 Monitor 1.5 (available from Roche Diagnostics) or of Nuclisens HIV-1 QT-1.

In certain embodiments, the formulations of the invention are useful to protect not only against sexual transmission of HIV, but also to prevent infection of a baby during passage through the birth canal. Thus the vaginal administration can take place prior to sexual intercourse, during sexual intercourse, immediately prior to childbirth or during childbirth. Such topical dosage forms may be particularly useful when applied to a newborn baby of an HIV-infected mother.

Thus, the present method may involve topical application to the vagina to prevent, slow or inhibit HIV infection as a result of vaginal intercourse. Typically, the topical application is carried out prior to the beginning of vaginal intercourse, suitably 0 to 60 minutes, preferably 0 to 5 minutes, prior to the beginning of vaginal intercourse. Suitably the formulation is applied in an amount that will result in a local concentration of 0.5 mM to 1M, preferably 0.5 mM to 500 mM, most preferably 25 mM to 50 mM, of the CCR5 antagonist(s) throughout the vagina. The higher concentrations provide a superior anti-HIV effect by interfering with the attachment of the virus to the CCR5 receptor.

Further, when only the female is infected with HIV, it is not necessary that the sperm be killed in order to prevent the sexual transmission of the disease. The formulation may be applied to the vagina in any conventional manner. Suitable devices for applying the composition to the vagina are disclosed in previously cited U.S. Pat. No. 5,989,581, as well as U.S. Pat. Nos. 3,826,828, 4,108,309, 4,360,013, and 4,589,880, which are incorporated herein by reference.

In another embodiment, the present invention involves topical administration of the topical formulation to the anus. Suitably, the formulation is applied in an amount which results in a local anal concentration of 0.5 mM to 1M, preferably 0.5 mM to 500 mM, most preferably 25 mM to 50 mM of the CCR5 antagonist(s). The composition administered to the anus is suitably a foam or gel, etc., such as those described above with regard to vaginal application. In the case of anal application, it may be preferred to use an applicator which distributes the composition substantially evenly throughout the anus. For example, a suitable applicator is a tube 2.5 to 25 cm, preferably 5 to 10 cm, in length having holes distributed regularly along its length.

When the composition is a water-soluble vaginal cream or gel, suitably 0.1 to 4 grams, preferably about 0.5 to 2 grams, are applied. When the composition is a vaginal spray-foam, suitably 0.1 to 2 grams, preferably about 0.5 to 1 grams, of the spray-foam are applied. When the composition is an anal cream or gel, suitably 0.1 to 4 grams, preferably about 0.5 to 2 grams of the cream or gel is applied. When the composition is an anal spray-foam, suitably 0.1 to 2 grams, preferably about 0.5 to 1 grams of the spray-foam are applied.

As a vaginal formulation, the active ingredient may be used in conjunction with a spermicide and may be employed with a condom, diaphragm, sponge or other contraceptive device. Examples of suitable spermicides include nonylphenoxypolyoxyethylene glycol (nonoxynol 9), benzethonium chloride, and chlorindanol. Suitably, the pH of the composition is 4.5 to 8.5. Vaginal compositions preferably have a pH of 4.5 to 6, most preferably about 5.

Vaginal formulations also include suppositories (for example, gel-covered creams), tablets and films. The suppositories can be administered by insertion with an applicator using methods well known in the art.

Typical buccal formulations are creams, ointments, gels, tablets or films that comprise ingredients that are safe when administered via the mouth cavity. Buccal formulations can also comprise a taste-masking or flavoring agent.

The present compositions may also be in the form of a time-release composition. In this embodiment, the CCR5 receptor antagonist is incorporated in a composition which will release the active compound at a rate which will result in the vaginal or anal concentration described above. Time-release compositions are disclosed in Controlled Release of Pesticides and Pharmaceuticals, D. H. Lew, Ed., Plenum Press, New York, 1981; and U.S. Pat. Nos. 5,185,155; 5,248,700; 4,011,312; 3,887,699; 5,143,731; 3,640,741; 4,895,724; 4,795,642; Bodmeier et al, Journal of Pharmaceutical Sciences, vol. 78 (1989); Amies, Journal of Pathology and Bacteriology, vol. 77 (1959); and Pfister et al, Journal of Controlled Release, vol. 3, pp. 229-233 (1986), all of which are incorporated herein by reference.

When a CCR5 antagonist formulation of the present invention is administered in combination with one or more separate formulations comprising another drug(s), e.g, one or more other antiviral compounds, the individual components of the combinations may be administered either sequentially or simultaneously in separate or combined pharmaceutical formulations. When a CCR5 antagonist formulation as described herein is used in combination with another active, the dose of the CCR5 antagonist and the other active(s) may be either the same or different from that when the CCR5 antagonist or the other active is used alone. The appropriate dose will be readily appreciated by those skilled in the art.

The present compositions may also be in the form which releases the CCR5 receptor antagonist in response to some event such as vaginal or anal intercourse. For example, the composition may contain the CCR5 receptor antagonist in vesicles or liposomes which are disrupted by the mechanical action of intercourse. Compositions comprising liposomes are described in U.S. Pat. No. 5,231,112 and Deamer and Uster, “Liposome Preparation: Methods and Mechanisms”, in Liposomes, pp. 27-51 (1983); Sessa et al, J. Biol. Chem., vol. 245, pp. 3295-3300 (1970); Journal of Pharmaceutics and Pharmacology, vol. 34, pp. 473-474 (1982); and Topics in Pharmaceutical Sciences, D. D. Breimer and P. Speiser, Eds., Elsevier, New York, pp. 345-358 (1985), which are incorporated herein by reference.

It should also be realized that the present compositions may be associated with a contraceptive device or article, such as a vaginal ring device, an intrauterine device (IUD), vaginal diaphragm, vaginal sponge, pessary, condom, etc. In the case of an IUD or diaphragm, time-release and/or mechanical-release compositions may be preferred, while in the case of condoms, mechanical-release compositions are preferred.

A suitable vaginal ring drug delivery system for slow release of the CCR5 antagonist is disclosed in U.S. Pat. No. 5,989,581, incorporated herein by reference. As described in U.S. Pat. No. 5,989,581, the vaginal ring delivers 2 actives for contraception. The drug delivery system disclosed comprises at least one compartment comprising a drug dissolved in a thermoplastic polymer core and a thermoplastic skin covering the core. Preferred thermoplastic polymers for both the core and the skin are ethylene-vinylacetate copolymers. As would be understood by one skilled in the art, according to the present invention, the disclosed delivery system contains at least one CCR5 antagonist useful to prevent, inhibit or slow infection or transmission of HIV. In certain embodiments, said vaginal ring device may also contain one or more additional drugs, for instance a contraceptive agent such as a steroidal progestogenic compound and/or a steroidal estrogenic compound. In yet other embodiments, the vaginal ring system containing a CCR5 antagonist may also contain or be used in combination with a topical estriol, such as Ovestin™, to enhance prevention of infection or transmission of HIV through the vaginal epithelium.

In another embodiment, the present invention provides novel articles which are useful for the prevention or retardation of HIV infection. In particular, the present articles are those which release the CCR5 receptor antagonist when placed on an appropriate body part or in an appropriate body cavity. Thus, the present article may be a vaginal ring device as described above or an IUD. Suitable IUDs are disclosed in U.S. Pat. Nos. 3,888,975 and 4,283,325 which are incorporated herein by reference.

The present article may be an intravaginal sponge which comprises and releases, in a time-controlled fashion, the CCR5 receptor antagonist. Intravaginal sponges are disclosed in U.S. Pat. Nos. 3,916,898 and 4,360,013, which are incorporated herein by reference. The present article may also be a vaginal dispenser which releases the CCR5 receptor antagonist. Vaginal dispensers are disclosed in U.S. Pat. No. 4,961,931, which is incorporated herein by reference.

The present article may also be a condom which is coated with the CCR5 receptor antagonist. In one embodiment, the condom is coated with a lubricant or penetration enhancing agent which comprises the CCR5 receptor antagonist. The lubricant or penetration enhancing agent can comprise the CCR5 receptor antagonist which is encapsulated in liposomes such that the CCR5 receptor antagonist is released from the liposomes upon intercourse. Lubricants and penetration enhancing agents are described in U.S. Pat. Nos. 4,537,776; 4,552,872; 4,557,934; 4,130,667, 3,989,816; 4,017,641; 4,954,487; 5,208,031; and 4,499,154, which are incorporated herein by reference. In another embodiment, the topical formulation of the present invention is contained inside the condom, for example in a reservoir in the tip of the condom.

For in vivo uses, the dose of CCR5 receptor antagonist administered to a human in the context of the present invention should be sufficient to affect a prophylactic or inhibitory response in the individual over a reasonable time frame. In particular embodiments, the dose of CCR5 antagonist should be in the range of 1-1000 mg per day or 1-1000 mg per application. The dose used to achieve a desired antiviral concentration in vivo (e.g., 0.1-1000 nM) will be determined by the potency of the particular CCR5 receptor antagonist employed.

### FORMULATION EXAMPLES

### Example 1

A vaginal cream formulation is prepared by mixing the components listed in Table 1 below. For each application, 1-4 grams of the cream are vaginally administered with a suitable applicator such as a syringe.

TABLE 1

| Component | Weight Percent |
|---|---|
| Compound Va | 10-40 |
| Cetyl esters wax | 1-15 |
| Cetyl alcohol | 2-5 |
| White wax | 5-20 |
| Glyceryl monostearate | 10-30 |
| Propylene glycol | 10-15 |
| monostearate | |
| Methyl stearate | 5-90 |
| Benzyl alcohol | 3-10 |
| Sodium lauryl sulfate | 0.5-2.5 |
| Glycerin | 5-30 |
| Mineral oil | 0.1-95 |

### Example 2

A vaginal cream formulation is prepared by mixing the components listed in Table 2 below. For each application, 1-4 grams of the cream are vaginally administered with a suitable applicator such as a syringe.

TABLE 2

| Component | Weight Percent |
|---|---|
| Compound IIIa | 10-40 |
| edetate disodium | 0.01-0.10 |
| glyceryl | 0.5-10 |
| monoisostearate | |
| methyl paraben | 0.18-0.20 |
| mineral oil | 0.1-95 |
| polyglyceryl-3-oleate | 2-3.5 |
| propylene glycol | 5-15 |
| propyl paraben | 0.02-0.10 |
| colloidal silicon dioxide | 1-5 |
| sorbitol solution | 2-18 |
| purified water | 10-20 |
| microcrystalline wax | 2-20 |

### Example 3

A vaginal gel formulation is prepared by mixing the components listed in Table 3 below. For each application, 4 grams of the gel are vaginally administered with a suitable applicator such as a syringe.

TABLE 3

| Component | Weight Percent |
|---|---|
| CCR5 antagonist of any | 10-40 |
| Formulas I-XI | |
| Carbomer 934P | 0.1-0.5 |
| Edetate disodium | 0.01-0.10 |
| Methyl paraben | 0.18-0.20 |
| Propyl paraben | 0.02-0.10 |
| Propylene glycol | 5-15 |
| Sodium hydroxide | 0.01-0.05 |

### Example 4

A rectal foam formulation is prepared by mixing the components listed in Table 4 below and inert propellants isobutene and propane. The foam is supplied in a aerosol container with a rectal applicator. For each application, 900 milligrams of the foam are rectally administered using the applicator.

TABLE 4

| Component | Weight Percent |
|---|---|
| CCR5 antagonist of any | 10-40 |
| Formulas I-XI | |
| Propylene glycol | 5-15 |
| Emulsifying wax | 10-15 |
| Polyoxyethylene-10- | 0.1-0.5 |
| stearyl ether | |
| Cetyl alcohol | 2-5 |
| Methyl paraben | 0.18-0.20 |
| Propyl paraben | 0.02-0.10 |
| Triethanolamine | 2-4 |
| Purified water | 10-30 |

### Example 5

The selective anti-viral activity of exemplary CCR5 Antagonist compounds was tested using the TZM-bi infection assay as described by Fletcher, et. al., 2009, Antimicrobial Agents & Chemother. 53 (2): 487-495 incorporated herein by reference in its entirety as modified below:

Cell and virus culture. All “complete” media were supplemented with 10% fetal calf serum, penicillin (100 U/ml), streptomycin (100 μg/ml), and L-glutamine (2 mM) unless otherwise stated. PM-1 cells (AIDS Reagent Project, NIBSC, United Kingdom) and MT-4 cells were grown in continual culture in complete RPMI medium. TZM-bI cells (NIH AIDS Research and Reference Reagent Program) were grown in continual culture in complete Dulbecco's modified Eagle medium and were treated with 1× trypsin-EDTA for cell passage. Primary human macrophages were prepared and purified from peripheral blood mononuclear cells and were cultured in complete RPMI medium containing 20% fetal calf serum.

Wild type strains of HIV-1, both CCR (R5) utilizing Wild type strains of HIV-1, Wild-type strains of HIV-1, both CCR5 (R5) utilizing (HIV-1_{BaL}) and CXCR4 (X4) utilizing (HIV-1_{RF} and HIV-1_{IIIB}), were grown either in phytohemagglutinin-stimulated peripheral blood mononuclear cells or in PM-1 cells.

(i) TZM-bI luciferase reporter assay. TZM-bI cells (5×10⁴/well) cultured overnight were treated with a range of compound dilutions for 1 h prior to exposure to HIV-1_{BaL} or HIV-1_{IIIB} (200 50% tissue culture infective doses [TCID₅₀]/ml). After 24 h, cells were washed and lysed, and luciferase units were determined using the luciferase assay kit (Stratagene, United Kingdom).

Determination of compound toxicity. The viability of cells (TZM-bI) and following treatment with the compound was determined by the principle of MTT dye reduction (see references cited in Fletcher et. al.). All data are expressed as the percentage of viability for compound-treated wells compared to untreated cell control wells, and the 50% cytotoxic concentration (CC₅₀) is defined as the concentration of the drug at which the cell/tissue viability was reduced to 50% of the drug-free control value.

Results obtained are shown in the Table below.

TABLE

| | Inhibition of HIV Infection | | |
|---|---|---|---|
| | (TZM-bL cells using luciferase reporter) | | Cytotoxicity |
| Compound | IC50-RS | IC50-X4 | CC50 (TZM-bL) |
| Compound 5L | 118.9 ± 92.4 nM (3) | ND | 476 ± 117 uM (3) |
| Tartrate | | | |
| Compound 28A | 2.4 ± 1.7 nM (3) | **Higher concentration | 73.5 ± 58.6 uM (5) |
| Hydrochloride | | may potentiate | |
| | | (around 62.6 uM) | |
| Compound 23 | 53.6 ± 45.1 nM (5) | **Higher concentration | 44.8 ± 11.5 uM (4) |
| Di-Hydrochloride | | may potentiate | |
| | | (around 62.6 uM) | |

Data represent mean ± D for (n = X) independent experiments, where each condition was tested in triplicate.

The results confirm the activity of the CCR 5 Antagonists.

Initial solubility assessments were made of Compounds 28A, 23 and 5L to evaluate compatibility in various solvents useful in making vaginal devices. The results obtained are shown in the Table below.

TABLE

| | | Solubility | | | |
|---|---|---|---|---|---|
| # | Solvent | Cmpd 5L | Cmpd 23 | Cmpd 28A | Note |
| 1 | DI H₂0 | ≧1.32 | ≧1.44 | ≧3.85 | |
| 2 | pH 4.4 citrate buffer | ≧12.6 | ≧15.2 | ≧11.4 | |
| 3 | pH 7.5 phosphate | ~0.75 | <0.24 | <0.33 | |
| | buffer | | | | |
| 5 | PG (propyleneglycol) | ≧45 (4.2%) | ≧56.2 (5.1%) | ≧38.4 (3.6%) | 1.038 g/cm\hat{ }3 |
| 6 | PEG 400 | ≧43.2 (3.7%) | ≧48.2 (4.1%) | ≧39 (3.4%) | 1.12 g/cm\hat{ }3 |

Overall, all three compounds exhibit fair solubility in citrate buffer at pH 4.4, and very good solubility in propylene glycol and PEG400.

For certain embodiments of the vaginal devices, it is believed that a crystalline form of a CCR5 Antagonist may be preferred. Solution based crystalline techniques were used and two anhydrous crystalline forms of compound 5L were isolated

Analytical techniques including powder X-ray diffraction (PXRD), differential scanning calorimetry (DSC), and thermogravimetric analysis (TGA) were utilized to characterize the solid-state properties of a number of CCR5 Antagonists including the glass transition temperature for amorphous materials, melting temperature for crystalline materials, and the onset temperature of thermal degradation.

Powder X-ray diffraction (PXRD) was performed on a Rigaku MiniFlex operated at 30 kV and 15 mA producing copper Kα radiation. The slit dimensions were variable for divergence and set to 4.2 deg for scattering and 0.3 mm for receiving. Data was collected from 2° to 35° 2θ with a sample interval of 0.02° and a rate of 2° per minute. Material was placed on either aluminum or silicon background holders for analysis.

Thermogravimetric analysis (TGA) was conducted with a TGA Q500 from TA instruments. A typical experiment involved heating the material in a platinum pan under a nitrogen purge from 25° C. to 350° C. at 10° C. per minute. Data was analyzed using the Universal using the Universal Analysis software program from TA Instruments.

Differential scanning colorimetry (DSC) was conducted with a DSC Q100 from TA Instruments. Approximately 1 to 2 mg of material was placed in an aluminum hermetic pan with 2 pinholes in the lid. Samples were heated under a nitrogen purge from 25° C. to a temperature near the onset of degradation as determined by TGA with a heating rate of 10° C. per minute. Data was analyzed using the Universal Analysis software program from TA Instruments.

Attempts to crystallize a compound involved weighing approximately 100 mg of material into a glass vial followed by adding 1 to 2 mL of solvent. If the material completely dissolved, the vial was covered with aluminum foil with one small hole poked in the foil to allow for slow solvent evaporation over the course of a week. If the material did not completely dissolve, a stir bar was added, the vial was capped, and the suspension was stirred for 10 days. After stirring, the stir bar was removed, the vial centrifuged, and the supernatant was discarded. Solids isolated from the evaporation and slurry experiments were analyzed for crystallinity by PXRD.

Results

Crystallinity.

Of the compounds investigated, only Compound 5L was able to be crystallized using these methods. For the remaining compounds, only amorphous material was recovered from each solvent. Solvents used included ethyl acetate, isopropanol, MTBE, acetone, methylene chloride, acetonitrile, tetrahydrofuran, n-heptane, water.

Crystalline material of Compound 5L was recovered from each solvent as determined by PXRD. Two crystalline forms (form 1 and form 2) were distinguished by PXRD and later by differential scanning calorimetry (DSC). The table below summarizes the results of the crystal form screen for Compound 5L.

TABLE
Summary of solvents and methods of crystallization for Compound 5L
Compound 5 L

| Solvent | Method | Result |
|---|---|---|
| ethyl acetate | evaporation | form 1 |
| Isopropanol | slurry | form 1 |
| MTBE | slurry | form 2 |
| acetone | evaporation | forms 1 + 2 |
| methylene chloride | evaporation | form 2 |
| acetonitrile | evaporation | form 1 |
| tetrahydrofuran | evaporation | form 1 |
| n-heptane | slurry | form 2 |
| water | slurry | form 2 |

Characterization

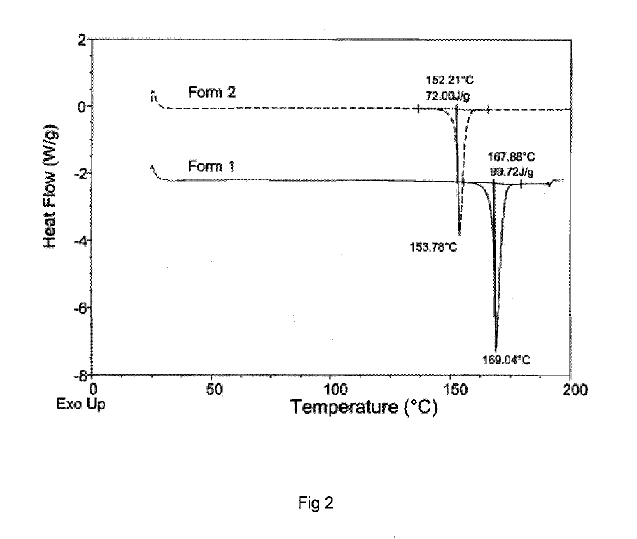

PXRD was used to identify two crystalline forms of Compound 5L (form 1 and form 2) and data for each form is shown in FIG. 1. Differential scanning calorimetry (DSC) was used to identify the melting point of each form and in some cases such as acetone, the material consisted of a mixture of both forms as evident by the PXRD patterns. The DSC traces for these mixtures show 2 endothermic events that correspond to melting of each form (Data not shown). In the cases of 2-proponal and MTBE, pure forms 1 and 2 were recovered as evident by the presence of only one melting endoderm in the DSC trace. The DSC thermograms for each form are shown in FIG. 2. Analysis of the DSC data indicate that form 1 has a melting point of 168° C. and enthalpy of fusion of 99.7 J/g and form 2 has a melting point of 152° C. and enthalpy of fusion of 72.0 J/g.

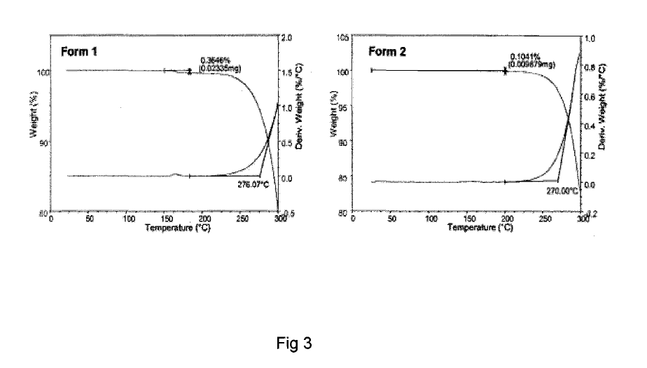

Thermogravimetric analysis (TGA) was performed on each form to evaluate the thermal stability and presence of any volatiles in the sample (FIG. 3). The onset of thermal degradation is estimated using the derivative of weigh loss curve and was measured as 276° C. and 270° C. for forms 1 and 2 respectively. The weight loss curve for form 1 shows a weight loss of 0.4% around 165° C. which is likely due to residual solvent trapped in the crystal that is liberated upon melting. Form 2 shows negligible weigh loss upon heating until thermal degradation.

Thermodynamic Stability of Forms 1 and 2

To assess the relative thermodynamic stability of forms 1 and 2, a competition slurry experiment was performed. In this experiment, the as received amorphous material was slurried in one vial containing 2-propanol and another containing MTBE. Seeds of both form 1 and form 2 were added to both vials and stirring continued for 1 week. The solid material was filtered from each vial and analyzed with PXRD and DSC. Material recovered from both vials was identified as pure form 1 by both PXRD and DSC therefore indicating that form 1 is the more thermodynamically stable form. This result is consistent with the DSC data and the heat of fusion rule which states that if the form with the higher melting temperature also has a greater heat of fusion, it is the more thermodynamically stable form and the two forms are related monotropically.

Glass Transition Temperature. The glass transition temperature (T_{g}) was measured using DSC. In a typical experiment, the amorphous material was placed in a DSC pan and heated to 200° C. under a nitrogen purge before cooing to a 0° C. and again heating to 200° C. The first cycle serves to remove any water or volatiles that may plasticize and reduce the T_{g} from its dry value. The second heating cycle was used to record the T_{g}. For Compound 5L, the crystalline material was melted by heating to 200° C. and then quenched to 0° C. yielding amorphous material before heating again to measure the glass transition temperatures (T_{g}) for compound 5L which was 64° C.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A topical formulation or a slow release device for vaginal or rectal administration comprising at least one small molecule CCR5 receptor antagonist which is
(i) a compound represented by the structural formula I: 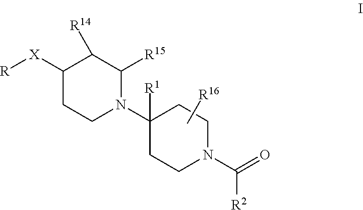
or a pharmaceutically acceptable salt thereof, wherein
X is —C(R¹³)₂—, —C(R¹³)(R¹⁹)—, —C(O)—, —O—, —NH—, —N((C₁-C₆)alkyl)-, 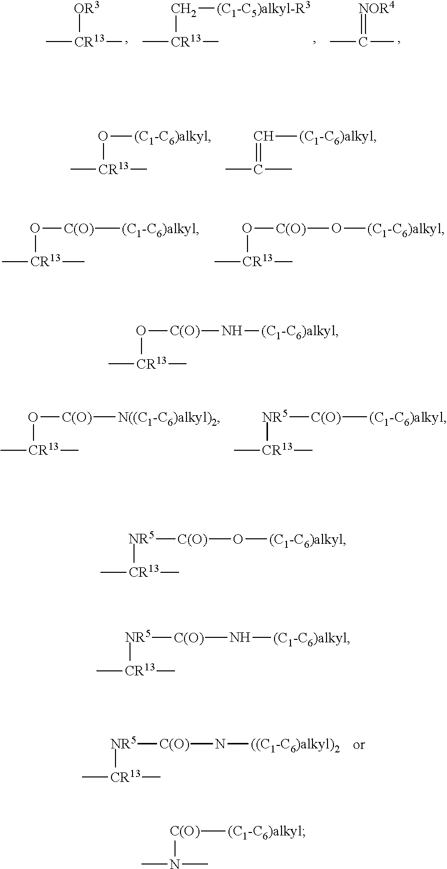
R is R⁶-phenyl, R⁶-pyridyl, R⁶-thiophenyl or R⁶-naphthyl;
R¹ is hydrogen, C₁-C₆ alkyl or C₂-C₆ alkenyl;
R² is R⁷, R⁸, R⁹-phenyl; R⁷, R⁸, R⁹-substituted 6-membered heteroaryl; R⁷, R⁸, R⁹-substituted 6-membered heteroaryl N-oxide; R¹⁰, R¹¹-substituted 5-membered heteroaryl; naphthyl; fluorenyl; diphenylmethyl 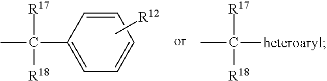
R³ is R⁶-phenyl, R⁶-heteroaryl or R⁶-naphthyl;
R⁴ is hydrogen, C₁-C₆ alkyl, fluoro-C₁-C₆ alkyl, cyclopropylmethyl, —CH₂CH₂OH, —CH₂CH₂—O—(C₁-C₆)alkyl, —CH₂C(O)—O—(C₁-C₆)alkyl, —CH₂C(O)NH₂, —CH₂C(O)—NH(C₁-C₆)alkyl or —CH₂C(O)—N((C₁-C₆)alkyl)₂;
R⁵ and R¹¹ are independently selected from the group consisting of hydrogen and (C₁-C₆)-alkyl;
R⁶ is 1 to 3 substituents independently selected from the group consisting of hydrogen, halogen, C₁-C₆ alkyl, C₁-C₆ alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃)—, CH₃C(═NOCH₂CH₃)—, 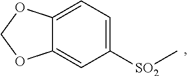
—NH₂, —NHCOCF₃, —NHCONH(C₁-C₆ alkyl), —NHCO(C₁-C₆ alkyl), —NHSO₂(C₁-C₆ alkyl), 5-membered heteroaryl and 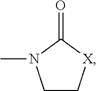
wherein X is —O—, —NH— or —N(CH₃)—;
R⁷ and R⁸ are independently selected from the group consisting of (C₁-C₆)alkyl, halogen, —NR²⁰R²¹, —OH, —CF₃, —OCH₃, —O-acyl, and —OCF₃;
R⁹ is R⁷, hydrogen, phenyl, —NO₂, —CN, —CH₂F, —CHF₂, —CHO, —CH═NOR²⁰, pyridyl, pyridyl N-oxide, pyrimidinyl, pyrazinyl, —N(R²⁰)CONR²¹R²², —NHCONH(chloro-(C₁-C₆)alkyl), —NHCONH(C₃-C₁₀)-cycloalkyl(C₁-C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂CF₃)₂, —NHCO₂(C₁-C₆)alkyl, C₃-C₁₀ cycloalkyl, —SR²³, —SOR²³, —SO₂R²³, —SO₂NH(C₁-C₅ alkyl), —OSO₂(C₁-C₆)alkyl, —OSO₂CF₃, hydroxy(C₁-C₆)alkyl, —CONR²⁰R²¹, —CON(CH₂CH₂—O—CH₃)₂,
—OCONH(C₁-C₆)alkyl, —CO₂R²⁶, —Si(CH₃)₃ or —B(OC(CH₃)₂)₂;
R¹⁰ is (C₁-C₆)alkyl, —NH₂ or R¹²-phenyl;
R¹² is 1 to 3 substituents independently selected from the group consisting of hydrogen, (C₁-C₆)alkyl, —CF₃, —CO₂R₂₀, —CN, (C₁-C₆)alkoxy and halogen;
R¹³, R¹⁴, R¹⁵ and R¹⁶ are independently selected from the group consisting of hydrogen and (C₁-C₆)alkyl;
R¹⁷ and R¹⁸ are independently selected from the group consisting of hydrogen and C₁-C₆ alkyl, or R¹⁷ and R¹⁸ together are a C₂-C₅ alkylene group and with the carbon to which they are attached form a spiro ring of 3 to 6 carbon atoms;
R¹⁹ is R⁶-phenyl, R⁶-heteroaryl, R⁶-naphthyl, C₃-C₁₀ cycloalkyl, (C₃-C₁₀)cycloalkyl(C₁-C₆)alkyl or (C₁-C₆)alkoxy(C₁-C₆)alkyl;
R²⁰, R²¹ and R²² are independently selected from the group consisting of H and C₁-C₆ alkyl; and
R²³ is C₁-C₆ alkyl or phenyl;
(ii) a compound represented by the structural formula II 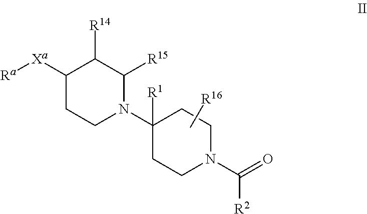
or a pharmaceutically acceptable salt thereof, wherein
(1) X^{a} is —C(R¹³)₂—, —C(R¹³)(R¹⁹)—, —C(O)—, —O—, —NH—, —N((C₁-C₆)alkyl)-, 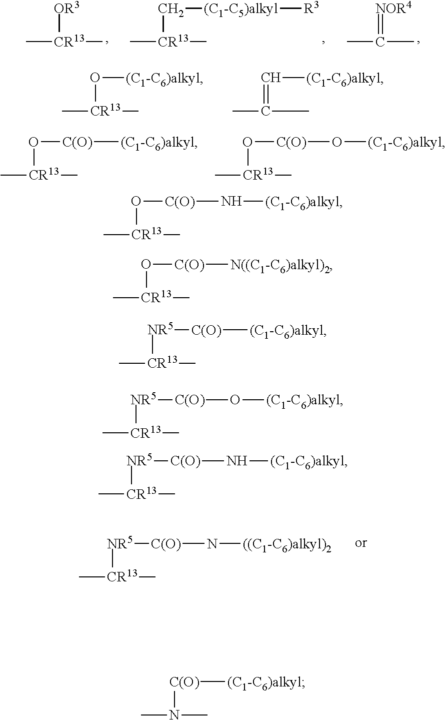
R^{a} is R^{6a}-phenyl, R^{6a}-pyridyl, R^{6a}-thiophenyl or R⁶-naphthyl;
R¹ is hydrogen, C₁-C₅ alkyl or C₂-C₆ alkenyl;
R² is R⁷, R⁸, R⁹-phenyl; R⁷, R⁸, R⁹-substituted 6-membered heteroaryl; R⁷, R⁸, R⁹-substituted 6-membered heteroaryl N-oxide; R¹⁰, R¹¹-substituted 5-membered heteroaryl; naphthyl; fluorenyl; diphenylmethyl 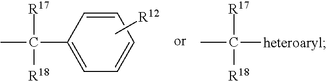
R³ is R¹⁰-phenyl, pyridyl, pyrimidyl, pyrazinyl or thiazolyl;
R⁴ is hydrogen, C₁-C₆ alkyl, fluoro-C₁-C₆ alkyl, cyclopropylmethyl, —CH₂CH₂OH, —CH₂CH₂—O—(C₁-C₆)alkyl, —CH₂C(O)—O—(C₁-C₆)alkyl, —CH₂C(O)NH₂, —CH₂C(O)—NH(C₁-C₆)alkyl or —CH₂C(O)—N((C₁-C₆)alkyl)₂;
R⁵ and R¹¹ are independently selected from the group consisting of hydrogen and (C₁-C₆)-alkyl;
R^{6a} is 1 to 3 substituents independently selected from the group consisting of hydrogen, halogen, —CF₃, CF₃O—, —CN, —CF₃SO₂—, R¹²-phenyl, —NHCOCF₃, 5-membered heteroaryl and 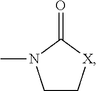
wherein X is —O—, —NH— or —N(CH₃)—;
R⁶ is independently selected from the group consisting of R^{6a} and CH₃SO₂—;
R⁷ and R⁸ are independently selected from the group consisting of (C₁-C₆)alkyl, halogen, —NR²⁰R²¹, —OH, —CF₃, —OCH₃, —O-acyl, and —OCF₃;
R⁹ is R⁷, hydrogen, phenyl, —NO₂, —CN, —CH₂F, —CHF₂, —CHO, —CH═NOR²⁰, pyridyl, pyridyl N-oxide, pyrimidinyl, pyrazinyl, —N(R²⁰)CONR²¹R²², —NHCONH(chloro-(C₁-C₆)alkyl), —NHCONH((C₃-C₁₀)-cycloalkyl(C₁-C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂CF₃)₂, —NHCO₂(C₁-C₆)alkyl, C₃-C₁₀ cycloalkyl, —SR²³, —SOR²³, —SO₂R²³, —SO₂NH(C₁-C₆)alkyl), —OSO₂(C₁-C₆)alkyl, —OSO₂CF₃, hydroxy(C₁-C₆)alkyl, —CONR²⁰R²¹, —CON(CH₂CH₂—O—CH₃)₂,
—OCONH(C₁-C₆)alkyl, —CO₂R²⁰, —Si(CH₃)₃ or —B(OC(CH₃)₂)₂;
R¹⁰ is (C₁-C₆)alkyl, —NH₂ or R¹²-phenyl;
R¹² is 1 to 3 substituents independently selected from the group consisting of hydrogen, (C₁-C₆)alkyl, —CF₃, —CO₂R₂₀, —CN, (C₁-C₆) alkoxy and halogen;
R¹³, R¹⁴, R¹⁵ and R¹⁶ are independently selected from the group consisting of hydrogen and (C₁-C₆) alkyl;
R¹⁷ and R¹⁸ are independently selected from the group consisting of hydrogen and C₁-C₆ alkyl, or R¹⁷ and R¹⁸ together are a C₂-C₅ alkylene group and with the carbon to which they are attached form a spiro ring of 3 to 6 carbon atoms;
R¹⁹ is R⁶-phenyl, R⁶-heteroaryl, R⁶-naphthyl, C₃-C₁₀ cycloalkyl, (C₃-C₁₀)cycloalkyl(C₁-C₆)alkyl or (C₁-C₆)alkoxy(C₁-C₆)alkyl;
R²⁰, R²¹ and R²² are independently selected from the group consisting of H and C₁-C₆ alkyl; and
R²³ is C₁-C₆ alkyl or phenyl;
or
(2): X^{a} is —C(R¹³)(R¹⁹)—, —C(O)—, —O—, —NH—, —N((C₁-C₆)alkyl)-, 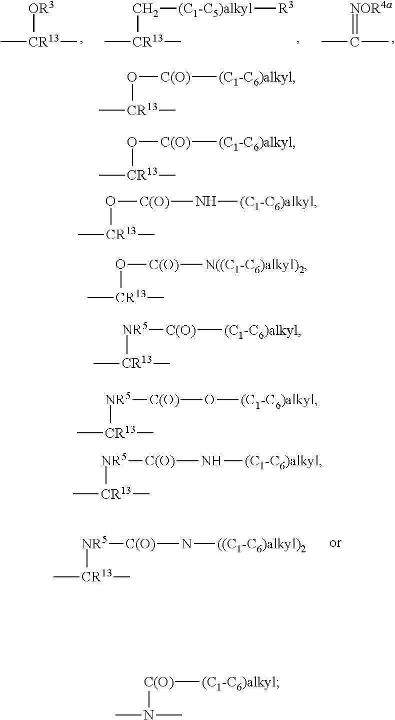
R^{a} is R^{6b}-phenyl, R^{6b}-pyridyl or R^{6b}-thiophenyl;
R^{4a} is fluoro-C₁-C₆ alkyl, cyclopropylmethyl, —CH₂CH₂OH, —CH₂CH₂—O—(C₁-C₆)alkyl, —CH₂C(O)—O—(C₁-C₆)alkyl, —CH₂C(O)NH₂, —CH₂C(O)—NH—(C₁-C₆)alkyl or —CH₂C(O)—N((C₁-C₆)alkyl)₂;
R^{6b} is CH₃SO₂—; and
R¹, R², R³, R⁵, R¹⁴, R¹⁵, R¹⁶ and R¹⁹ are as defined in (1) above;
(iii) a compound represented by the structural formula III: 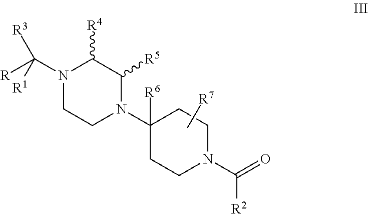
or a pharmaceutically acceptable salt thereof, wherein
R is R⁸-phenyl, R⁸-pyridyl, R⁸-thiophenyl or R⁸-naphthyl;
R¹ is hydrogen or C₁-C₆ alkyl;
R² is R⁹, R¹⁰, R¹¹-phenyl; R⁹,R¹⁰, R¹¹-substituted 6-membered heteroaryl; R⁹, R¹⁰, R¹¹-substituted 6-membered heteroaryl N-oxide; R¹², R¹³-substituted 5-membered heteroaryl; naphthyl; fluorenyl; diphenylmethyl 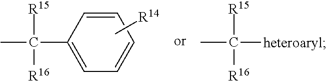
R³ is hydrogen, C₁-C₆ alkyl, (C₁-C₆)alkoxy(C₁-C₆)alkyl, C₃-C₁₀ cycloalkyl, C₃-C₁₀ cycloalkyl(C₁-C₆)alkyl, R⁸-phenyl, R⁸-phenyl(C₁-C₆)alkyl, R⁸-naphthyl, R⁸-naphthyl(C₁-C₆)alkyl, R⁸-heteroaryl or R⁸-heteroaryl(C₁-C₆)alkyl;
R⁴, R⁵, R⁷ and R¹³ are independently selected from the group consisting of hydrogen and (C₁-C₆)-alkyl;
R⁶ is hydrogen, C₁-C₆ alkyl or C₂-C₆ alkenyl;
R⁸ is 1 to 3 substituents independently selected from the group consisting of hydrogen, halogen, C₁-C₆ alkyl, C₁-C₆ alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃), CH₃C(═NOCH₂CH₃), 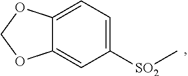
—NH₂, —NHCOCF₃,
—NHCONH(C₁-C₆ alkyl), —NHCO(C₁-C₆ alkyl), —NHSO₂(C₁-C₆ alkyl), 5-membered heteroaryl and 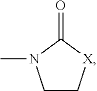
wherein X is —O—, —NH— or —N(CH₃)—;
R⁹ and R¹⁰ are independently selected from the group consisting of (C₁-C₆)alkyl, halogen, —NR¹⁷R¹⁸, —OH, —CF₃, —OCH₃, —O-acyl, —OCF₃ and —Si(CH₃)₃;
R¹¹ is R⁹, hydrogen, phenyl, —NO₂, —CN, —CH₂F, —CHF₂, —CHO, —CH═NOR¹⁷, pyridyl, pyridyl N-oxide, pyrimidinyl, pyrazinyl, —N(R¹⁷)CONR¹⁸R¹⁹, —NHCONH(chloro-(C₁-C₆)alkyl), —NHCONH((C₃-C₁)cycloalkyl(C₁-C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂CF₃)₂, —NHCO₂(C₁-C₆)alkyl, C₃-C₁₀ cycloalkyl, —SR²⁰, —SOR²⁰, —SO₂R²⁰, —SO₂NH(C₁-C₆ alkyl), —OSO₂(C₁-C₆)alkyl, —OSO₂CF₃, hydroxy(C₁-C₆)alkyl, —CONR¹⁷R¹⁸, —CON(CH₂CH₂—O—CH₃)₂, —OCONH(C₁-C₆)alkyl, —CO₂R¹⁷, —Si(CH₃)₃ or —B(OC(CH₃)₂)₂;
R¹² is (C₁-C₆)alkyl, —NH₂ or R¹⁴-phenyl;
R¹⁴ is 1 to 3 substituents independently selected from the group consisting of hydrogen, (C₁-C₆)alkyl, —CF₃, —CO₂R₁₇, —CN, (C₁-C₆)alkoxy and halogen;
R¹⁵ and R¹⁶ are independently selected from the group consisting of hydrogen and C₁-C₆ alkyl, or R¹⁵ and R¹⁶ together are a C₂-C₅ alkylene group and with the carbon to which they are attached form a spiro ring of 3 to 6 carbon atoms;
R¹⁷, R¹⁸ and R¹⁹ are independently selected from the group consisting of H and C₁-C₆ alkyl; and
R²⁰ is C₁-C₆ alkyl or phenyl;
(iv) a compound represented by the structural formula IV 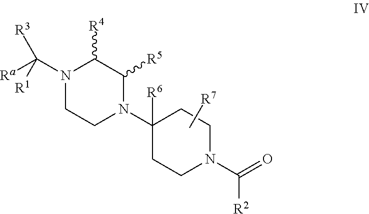
or a pharmaceutically acceptable salt thereof, wherein
(1) R^{a} is R^{8a}-phenyl, R^{8b}-pyridyl, R^{8b}-thiophenyl or R⁸-naphthyl;
R¹ is hydrogen or C₁-C₆ alkyl;
R² is R⁹, R¹⁰, R¹¹-phenyl; R⁹,R¹⁰,R¹¹-substituted 6-membered heteroaryl; R⁹, R¹⁰, R¹¹-substituted 6-membered heteroaryl N-oxide; R¹², R¹³-substituted 5-membered heteroaryl; naphthyl; fluorenyl; diphenylmethyl, 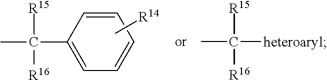
R³ is hydrogen, C₁-C₆ alkyl, (C₁-C₆)alkoxy(C₁-C₆)alkyl, C₃-C₁₀ cycloalkyl, C₃-C₁₀ cycloalkyl(C₁-C₆)alkyl, R⁸-phenyl, R⁸-phenyl(C₁-C₆)alkyl, R⁸-naphthyl, R⁸-naphthyl(C₁-C₆)alkyl, R⁸-heteroaryl or R⁸-heteroaryl(C₁-C₆)alkyl;
R⁴, R⁵, R⁷ and R¹³ are independently selected from the group consisting of hydrogen and (C₁-C₆)-alkyl;
R⁶ is hydrogen, C₁-C₆ alkyl or C₂-C₆ alkenyl;
R⁸ is 1 to 3 substituents independently selected from the group consisting of hydrogen, halogen, C₁-C₆ alkyl, C₁-C₆ alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃), CH₃C(═NOCH₂CH₃), 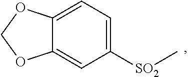
—NH₂, —NHCOCF₃, —NHCONH(C₁-C₆ alkyl), —NHCO(C₁-C₆ alkyl), —NHSO₂(C₁-C₆ alkyl), 5-membered heteroaryl and 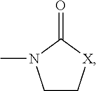
wherein X is —O—, —NH— or —N(CH₃)—;
R^{8a} is 1 to 3 substituents independently selected from the group consisting of hydrogen, halogen, —CF₃, CF₃O—, —CN, CF₃SO₂—, R¹⁴-phenyl, —NHCOCF₃, 5-membered heteroaryl and 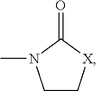
wherein X is as defined above;
R^{8b} is 1 to 3 substituents independently selected from the group consisting of hydrogen, halogen, —CF₃, CF₃O—, CH₃C(O)—, —CN, CF₃SO₂—, R¹⁴-benzyl, CH₃C(═NOCH₃), CH₃C(═NOCH₂CH₃), 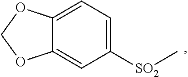
—NHCOCF₃, 5-membered heteroaryl and 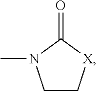
wherein X is as defined above;
R⁹ and R¹⁰ are independently selected from the group consisting of (C₁-C₆)alkyl, halogen, —NR¹⁷R¹⁸, —OH, —CF₃, —OCH₃, —O-acyl, —OCF₃ and —Si(CH₃)₃;
R¹¹ is R⁹, hydrogen, phenyl, —NO₂, —CN, —CH₂F, —CHF₂, —CHO, —CH═NOR¹⁷, pyridyl, pyridyl N-oxide, pyrimidinyl, pyrazinyl, —N(R¹⁷)CONR¹⁸R¹⁹, —NHCONH(chloro-(C₁-C₆)alkyl), —NHCONH(C₃-C₁)cycloalkyl(C₁-C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂CF₃)₂, —NHCO₂(C₁-C₆)alkyl, C₃-C₁₀ cycloalkyl, —SR²⁰, —SOR²⁰, —SO₂R²⁰, —SO₂NH(C₁-C₆ alkyl), —OSO₂(C₁-C₆)alkyl, —OSO₂CF₃, hydroxy(C₁-C₆)alkyl, —CONR¹⁷R¹⁸, —CON(CH₂CH₂—O—CH₃)₂,
—OCONH(C₁-C₆)alkyl, —CO₂R¹⁷, —Si(CH₃)₃ or —B(OC(CH₃)₂)₂;
R¹² is (C₁-C₆)alkyl, —NH₂ or R¹⁴-phenyl;
R¹⁴ is 1 to 3 substituents independently selected from the group consisting of hydrogen, (C₁-C₆)alkyl, —CF₃, —CO₂R₁₇, —ON, (C₁-C₆) alkoxy and halogen;
R¹⁵ and R¹⁶ are independently selected from the group consisting of hydrogen and
C₁-C₆ alkyl, or R¹⁵ and R¹⁶ together are a C₂-C₅ alkylene group and with the carbon to which they are attached form a spiro ring of 3 to 6 carbon atoms;
R¹⁷, R¹⁸ and R¹⁹ are independently selected from the group consisting of H and C₁-Cₛ alkyl; and
R²⁰ is C₁-C₆ alkyl or phenyl; or
(2) R^{a} is R⁸-phenyl, R⁸-pyridyl or R⁸-thiophenyl;
R² is fluorenyl, diphenylmethyl, 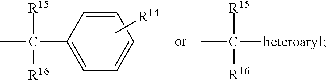
and R¹, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰ are as defined in (1); or
(v) a compound represented by the structural formula V 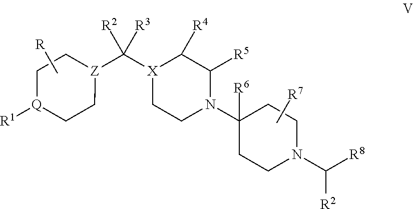
or a pharmaceutically acceptable salt or isomer thereof, wherein:
Q, X and Z are independently selected from the group consisting of CH and N,
provided that one or both of Q and Z is N;
R, R⁴, R⁵, R⁶ and R⁷ are independently selected from the group consisting of H and (C₁-C₆)alkyl;
R¹ is H, (C₁-C₆)alkyl, fluoro-(C₁-C₆)alkyl-, R⁹-aryl(C₁-C₆)alkyl-, R⁹-heteroaryl-(C₁-C₆)alkyl-, (C₁-C₆)alkyl-SO₂—, (C₃-C₆)cycloalkyl-SO₂—, fluoro-(C₁-C₆)alkyl-SO₂—, R⁹-aryl-SO₂—, R⁹-heteroaryl-SO₂—, N(R²²)(R²³)—SO₂—, (C₁-C₆)alkyl-C(O)—, (C₃-C₆)cyclo-alkyl-C(O)—, fluoro-(C₁-C₆)alkyl-C(O)—, R⁹-aryl-C(O)—, NH—(C₁-C₆)alkyl-C(O)— or R⁹-aryl-NH—C(O)—;
R² is H or (C₁-C₆)alkyl, and R³ is H, (C₁-C₆)alkyl, (C₁-C₆)alkoxy(C₁-C₆)alkyl-, (C₃-C₁₀)-cycloalkyl-, (C₃-C₁₀)cycloalkyl(C₁-C₆)alkyl-, R⁹-aryl, R⁹-aryl(C₁-C₆)-alkyl-, R⁹-heteroaryl, or R⁹-heteroaryl(C₁-C₆)alkyl-, provided that both X and Z are not each N;
or R² and R³ together are ═O, ═NOR¹⁹, ═N—NR¹¹R¹² or ═CH(C₁-C₆)alkyl, provided that when one or both of X and Z is N, R² and R³ together are not ═CH(C₁-C₆)alkyl;
and when X and Z are each CH, R³ can also be (C₁-C₆)alkoxy, R⁹-aryloxy, R⁹-heteroaryloxy, (C₁-C₆)alkyl-C(O)O—, (C₁-C₆)alkyl-NH—C(O)O—, N((C₁-C₆)alkyl)₂-C(O)O—, (C₁-C₆)alkyl-C(O)—NR¹³—, (C₁-C₆)alkyl-O—C(O)—NR¹³—, (C₁-C₆)alkyl-NH—C(O)—NR¹³— or N((C₁-C₆)alkyl)₂-C(O)—NR¹³—;
R⁸ is (R¹⁴,R¹⁵,R¹⁶)-substituted phenyl, (R¹⁴,R¹⁵,R¹⁶)-substituted 6-membered heteroaryl, (R¹⁴R¹⁵,R¹⁶)-substituted 6-membered heteroaryl N-oxide, (R¹⁷,R¹⁸)-substituted 5-membered heteroaryl, naphthyl, fluorenyl, diphenylmethyl, 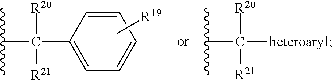
R⁹ is 1, 2 or 3 substituents independently selected from the group consisting of H, halogen, (C₁-C₆)alkyl, (C₁-C₆)alkoxy, —CF₃, —OCF₃, CH₃C(O)—, —CN, CH₃SO₂—, CF₃SO₂— and —N(R²²)(R²³);
R¹⁰ is H, (C₁-C₆)alkyl, fluoro(C₁-C₆)alkyl-, (C₃-C₁₀)cycloalkyl(C₁-C₆)alkyl-, hydroxy(C₂-C₆)alkyl-, (C₁-C₆)alkyl-O—(C₂-C₆)alkyl-, (C₁-C₆)alkyl-O—C(O)—(C₁-C₆)alkyl- or N(R²²)(R²³)—C(O)—(C₁-C₆)alkyl-;
R¹¹ and R¹² are independently selected from the group consisting of H, (C₁-C₆)alkyl and (C₃-C₁₀)cycloalkyl, or R¹¹ and R¹² together are C₂-C₆ alkylene and form a ring with the nitrogen to which they are attached;
R¹⁴ and R¹⁵ are independently selected from the group consisting of (C₁-C₆)alkyl, halogen, —NR²²R²³, —OH, —CF₃, —OCH₃, —O-acyl and —OCF₃;
R¹⁶ is R¹⁴, hydrogen, phenyl, —NO₂, —CN, —CH₂F, —CHF₂, —CHO, —CH═NOR²⁴, pyridyl, pyridyl N-oxide, pyrimidinyl, pyrazinyl, —N(R²⁴)CONR²⁵R²⁶, —NHCONH(chloro-(C₁-C₆)alkyl), —NHCONH((C₃-C₁₀)cycloalkyl(C₁-C₆)alkyl), —NHCO(C₁-C₆)alkyl, —NHCOCF₃, —NHSO₂N(R²²)(R²³), —NHSO₂(C₁-C₆)alkyl, —N(SO₂CF₃)₂, —NHCO₂—(C₁-C₆)alkyl, C₃-C₁₀ cycloalkyl, —SR²⁷, —SOR²⁷, —SO₂R²⁷, —SO₂NH(R²²), —OSO₂(C₁-C₆)alkyl, —OSO₂CF₃, hydroxy(C₁-C₆)alkyl-, —CONR²⁴R²⁵, —CON(CH₂CH₂OCH₃)₂, —OCONH(C₁-C₆)alkyl, —CO₂R²⁴, —Si(CH₃)₃ or —B(OC(CH₃)₂)₂;
R¹⁷ is (C₁-C₆)alkyl, —N(R²²)(R²³) or R¹⁹-phenyl;
R¹³, R¹⁸, R²², R²³, R²⁴, R²⁵ and R²⁶ are independently selected from the group consisting of H and (C₁-C₆)alkyl;
R¹⁹ is 1, 2 or 3 substituents independently selected from the group consisting of H, (C₁-C₆)alkyl, —CF₃, —CO₂R²⁵, —CN, (C₁-C₆)alkoxy and halogen;
R²⁰ and R²¹ are independently selected from the group consisting of H and (C₁-C₆)alkyl, or R²⁰ and R²¹ together with the carbon to which they are attached form a Spiro ring of 3 to 6 carbon atoms; and
R²⁷ is (C₁-C₆)alkyl or phenyl, and a pharmaceutically acceptable carrier.

2. The formulation of claim 1 comprising:
(a) at least one CCR5 antagonist of Formula I to XI;
(b) at least one lubricant; and
(c) at least one adjuvant where the adjuvant is an antimicrobial agent, antioxidant, humectant, or emulsifier, or a mixture of two or more thereof.

3-7. (canceled)

8. The topical formulation according to claim 1 wherein the at least one CCR5 receptor antagonist is 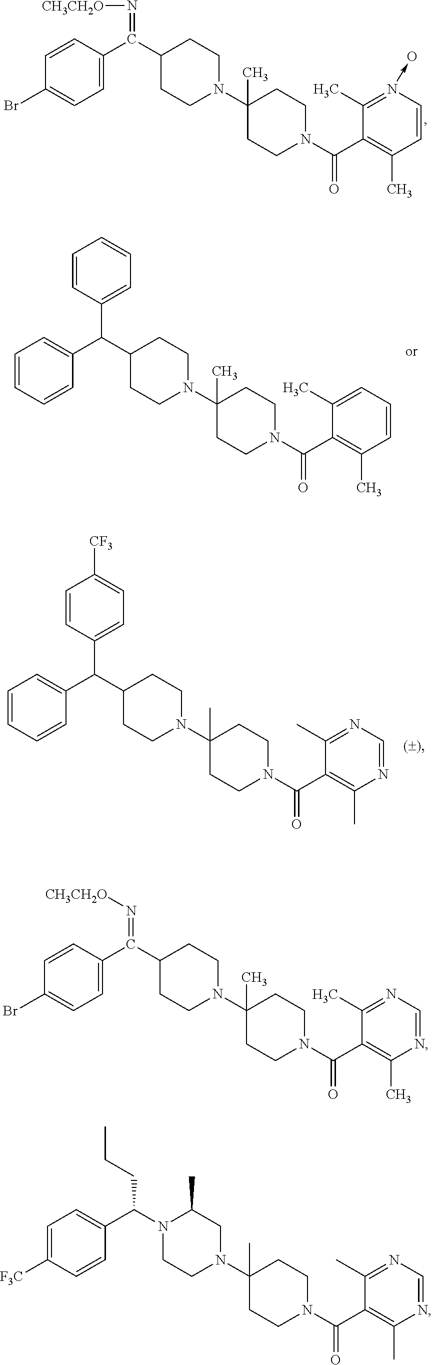 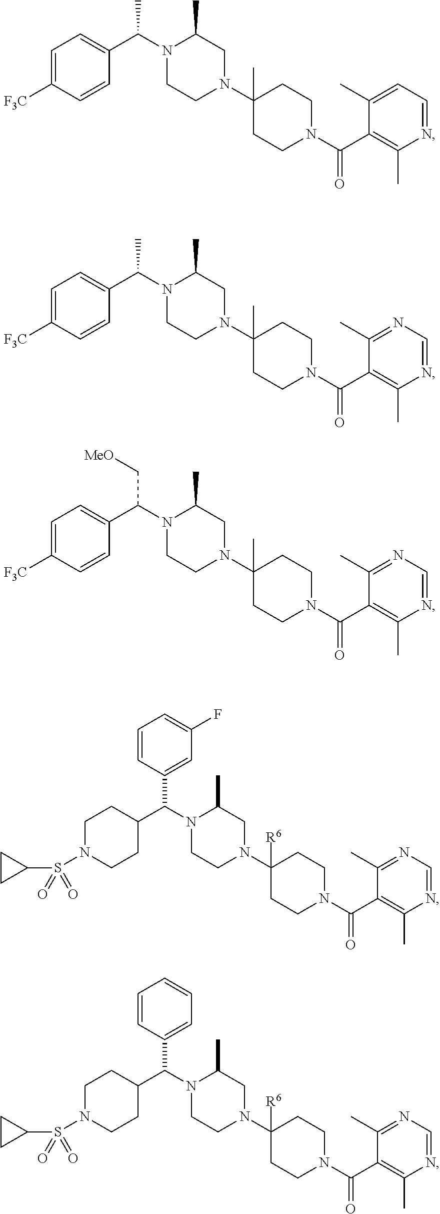 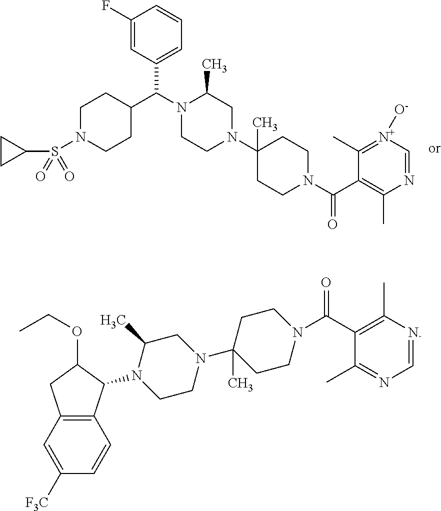
or a pharmaceutically acceptable salt of any of said compounds.

9. A topical formulation or a slow release device for vaginal or rectal administration comprising CCR5 Antagonist which is 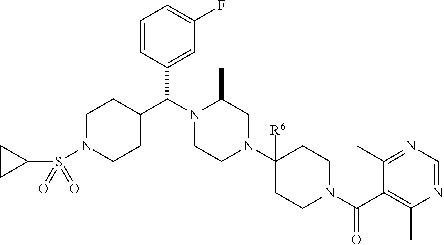
or a pharmaceutically acceptable salt thereof and a pharmaceutically acceptable carrier.

10. A topical formulation or a slow release device for vaginal or rectal administration comprising CCR5 Antagonist which is 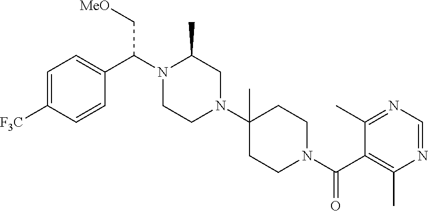
or a pharmaceutically acceptable salt thereof and a pharmaceutically acceptable carrier.

11. The topical formulation according to claim 2, wherein the lubricant is cetyl esters wax, hydrogenated vegetable oil, magnesium stearate, methyl stearate, mineral oil, polyoxyethylene-polyoxypropylene copolymer, polyethylene glycol, polyvinyl alcohol, sodium lauryl sulfate or white wax, or a mixture of two or more thereof.

12. The topical formulation according to any of claims 2, wherein the antimicrobial agent is propylene glycol, methyl paraben or propyl paraben, or a mixture of two or more thereof.

13. The topical formulation according to claim 2, wherein the formulation comprises at least one antioxidant.

14. The topical formulation according claim 13, wherein the antioxidant is butylated hydroxyanisole, butylated hydroxytoluene, or edetate disodium, or a mixture of two or more thereof.

15. The topical formulation according to claim 2, wherein the formulation comprises at least one humectant.

16. The topical formulation according to claim 15, wherein the humectant is ethylene glycol, glycerin, or sorbitol, or a mixture of two or more thereof.

17. The topical formulation according to claim 2, wherein the formulation comprises at least one emulsifier.

18. The topical formulation according to claim 2, wherein the emulsifier is carbomer, polyoxyethylene-10-stearyl ether, polyoxyethylene-20-stearyl ether, cetostearyl alcohol, cetyl alcohol, cholesterol, diglycol stearate, glyceryl monostearate, glyceryl stearate, hydroxypropyl cellulose, hydroxypropylmethyl cellulose, lanolin, polyoxyethylene lauryl ether, methyl cellulose, polyoxyethylene stearate, polysorbate, propylene glycol monostearate, sorbitan esters or stearic acid, or a mixture of two or more thereof.

19. The topical formulation according to claim 1, wherein the formulation is a vaginal cream comprising at least one CCR5 receptor antagonist, cetyl esters wax, cetyl alcohol, white wax, glyceryl monostearate, propylene glycol monostearate, methyl stearate, benzyl alcohol, sodium lauryl sulfate, glycerin and mineral oil.

20. The topical formulation according to claim 1, wherein the formulation is a vaginal cream comprising at least one CCR5 receptor antagonist, edentate disodium, glyceryl monoisostearate, methyl paraben, mineral oil, polyglyceryl-3-oleate, propylene glycol, propyl paraben, colloidal silicon dioxide, sorbitol solution, purified water and microcrystalline wax.

21. The topical gel formulation according to claim 1, wherein the formulation is a vaginal gel comprising at least one CCR5 receptor antagonist, carbomer, edentate disodium, methyl paraben, propyl paraben, propylene glycol, and sodium hydroxide.

22. The formulation according to claim 1, wherein the formulation is a rectal foam formulation comprising at least one CCR5 receptor antagonist, propylene glycol, emulsifying wax, polyoxyethylene-10-stearyl ether, cetyl alcohol, methyl paraben, propyl paraben, triethanolamine, purified water, and inert propellants isobutene and propane.

23. The slow release device according to claim 1 which is a vaginal ring device.

24-25. (canceled)

26. A method of slowing or inhibiting transmission of Human Immuno deficiency Virus comprising administering to a human in need thereof an effective amount of a formulation claim 1.

27. (canceled)

28. The method according to claim 26, further comprising
administering an effective amount of a one or more other antiviral or other agents useful in the treatment of Human Immuno-deficiency Virus that is/are different from the CCR5 receptor antagonist.

29. (canceled)
